(19) United States
(12) Patent Application Publication
(10) Pub. No.: US 2011/0091384 A1
(43) Pub. Date: Apr. 21, 2011

(54) Biomarker for identification of melanoma tumor cells

(75) Inventors: Rhoda M. Alani, Newton, MA (US); Whei F. Moriarty, Odenton, MD (US)

(73) Assignee: The Johns Hopkins University, Baltimore, MD (US)

(21) Appl. No.: 12/923,884

(22) Filed: Oct. 13, 2010

Related U.S. Application Data
(60) Provisional application No. 61/251,123, filed on Oct. 13, 2009.
(60) Provisional application No. 61/257,074, filed on Nov. 2, 2009.

(51) Int. Cl. A61K 49/00; G01N 33/68; A61K 39/395; C07K 16/30; A61P 35/00

(52) U.S. Cl. 424/9.1; 435/7.1; 435/7.23; 436/501; 436/64; 424/174.1; 530/389.7

(57) ABSTRACT

This invention relates to use of neuropilin-2 as a novel biomarker and therapeutic target for melanoma. The presence of neuropilin-2 can be used as a biomarker for diagnosing and detecting individuals suffering from or at risk for developing melanoma. Also described are methods of using neuropilin-2 to capture circulating melanoma cells. The present invention further relates to methods of treating an individual suffering from or at risk for developing melanoma with an agent that inhibits the activity of neuropilin-2.

This application claims the benefit of the filing date of provisional patent application Nos. 61/251,123, filed Oct. 13, 2009, and 61/257,074, filed Nov. 2, 2009, which are incorporated by reference in their entirety herein.

### FIELD OF INVENTION

The invention relates to use of neuropilin-2, a cell surface receptor, as a novel biomarker in the detection of melanoma tumor cells and as a novel therapeutic target in the treatment of melanomas.

### BACKGROUND INFORMATION

Melanoma incidence is increasing at one of the fastest rates for all cancers in the United States with a current lifetime risk of 1 in 58. In the United States in 2008, over 60,000 patients are expected to be diagnosed with melanoma with more than 8,000 deaths. There are currently no effective systemic therapies for late stage disease, and the average lifetime expectancy for patients with advanced melanoma is 6-9 months. Diagnosis of melanoma can be difficult as there is histologic overlap between benign and malignant lesions which can lead to both over and under diagnosis. In addition, determining the prognosis for a particular patient using current clinical criteria may be imprecise. The most useful prognostic indicators of primary cutaneous melanomas are Breslow depth and presence or absence of ulceration. However, many patients with thick melanomas are free of metastasis, while others with thin tumors die early from their disease. Despite numerous investigations to date, there are currently no adequate methods to accurately identify which melanomas will progress to vertical growth and metastasis.

### DESCRIPTION OF THE DRAWINGS

The patent or application file contains at least one drawing executed in color. Copies of this patent or patent application publication with color drawing(s) will be provided by the Office upon request and payment of the necessary fee.

FIG. 1. Methods of controlled heterotypic cell co-culture allow analysis of the cell-cell communication phenotype. FIG. 1A shows three methods for co-culturing heterotypic cells. In the gap interface method (I), RFP-HUVEC and GFP-1205Lu metastatic melanoma cells are co-cultured in colonies separated by a precisely defined gap. In the random mixture method (II), RFP-HUVEC and GFP-1205Lu metastatic melanoma cells are mixed together and cultured as a heterogeneous population of cells. In the micropatterning method (III), an RFP-HUVEC colony of precisely defined size and shape is surrounded by GFP-1205Lu cells. FIG. 1B shows the phenotype of cells co-cultured using the gap interface method at 6 hrs. (upper panels) and 48 hrs. (lower panel). FIG. 1C shows the phenotypes of cells co-cultured using the random mixture method at 6 hrs. (upper panels) and 48 hrs. (lower panel). FIG. 1D shows the phenotypes of the cells co-cultured using the micropatterning method at 6 hrs. (upper panels) and 48 hrs. (lower panel). FIG. 1E shows RFP-HUVECs cultured alone in EGM-2 medium for 48 hrs. FIG. 1F shows RFP-HUVECs cultured alone in conditioned medium (CM) from HUVECs for 48 hrs. FIG. 1G shows RFP-HUVECs co-cultured with 1205Lu cells for 48 hrs. FIG. 1H shows RFP-HUVECs co-cultured with GFP-1205Lu cells for 48 hrs.

FIG. 2. Global gene expression profiling of melanoma-endothelial cell interactions identifies NRP2 as a mediator of cellular communication. FIG. 2A is a schematic representation of a screening to identify genes involved in melanoma-endothelial cell communication. Using the random mixture method, populations of RFP-HUVECs and GFP-1205Lu metastatic melanoma cells were plated in a co-culture system and incubated for 48 hours. Cells were sorted by FACS, and RNAs were isolated and hybridized to a pan-genomic human GeneChip. Expression profiles altered by co-culture were compared against those in homotypic cultures of RFP-HUVECs and GFP-1205Lu cells to identify genes associated with melanoma-endothelial cell communication. FIG. 2B shows the western blot results for NRP2 expression in GFP-1205Lu cells grown in homotypic cell culture or following heterotypic co-culture with RFP-HUVECs. FIG. 2C shows the immunohistochemical staining results for NRP2 in human melanoma metastases at low-power magnification. FIGS. 2D and 2E show the immunohistochemical staining results for NRP2 in human melanoma metastases at high-power magnification. Arrows point to the demarcation of the tumor-metastatic niche interface. FIG. 2F shows a Melan-A stain for the melanoma cells depicted in FIG. 2E, demonstrating the correlation of NRP2 and Melan-A staining in serial tumor sections.

FIG. 3. Neutralizing antibody to NRP2 blocks metastatic melanoma cell proliferation. FIGS. 3A-3C show the proliferation assay for GFP-1205Lu metastatic melanoma cells in the presence of 10 μg/ml (FIG. 3A), 5 μg/ml (FIG. 3B), or 2.5 μg/ml (FIG. 3C) normal rabbit IgG (open circle) or rabbit polyclonal NRP2 antibody (closed circle). FIG. 3D shows the quantification of BrdU incorporation in GFP-1205Lu cells following 48 hours of treatment with 10 μg/ml NRP2 neutralizing antibody versus control antibody. Error bars represent standard deviation. *p<0.05, **p<0.01. FIG. 3E shows the results for a proliferation assay with GFP-1205Lu metastatic melanoma cells in the presence of 10 μg/ml normal mouse IgG or mouse monoclonal NRP2 antibody (sc-2025). FIGS. 3F-H show the results for a TUNEL assay with 1205Lu melanoma cells treated with 10 μg/ml normal rabbit IgG (FIG. 3F), rabbit polyclonal NRP2 antibody (FIG. 3G), or DNase positive control (FIG. 3H). FIGS. 3I and 3J show the phenotype of GFP-1205Lu cells following 48 hours of treatment with normal rabbit IgG (FIG. 3I) or NRP2 neutralizing antibody (FIG. 3J). FIG. 3K shows the results of a scratch assay performed on 1205Lu melanoma cells in the presence of 10 μg/ml normal rabbit IgG or rabbit polyclonal NRP2 antibody with and without Mitomycin C.

FIG. 4. Analysis of collective cell movements within HUVEC colonies of defined initial geometry, cell number, and size suggests NRP2's essential role in promotion of cellular patterning. FIG. 4A shows the expansion of a circular HUVEC alone colony in the absence of melanoma cells at T=0, 5, and 40 hrs. FIG. 4B shows the expansion of a circular HUVEC colony surrounded by a monolayer of GFP-1205Lu cells in the presence of NRP2-neutralizing antibody at T=0, 5, and 40 hrs. FIGS. 4C-4E show the results of quantitative analysis of collective cell movements from HUVEC colonies of defined geometry, cell number, and size for HUVEC island alone (FIG. 4C), HUVEC island and melanoma co-culture (FIG. 4D), and HUVEC island and melanoma co-culture treated with NRP2-neutralizing antibody (FIG. 4E). Three independent co-culture experiments were performed for each condition. The error bars represent the standard error of the mean.

FIG. 5. Analysis of NRP2 receptor and ligand expression in melanomas. FIG. 5A shows the quantitative expression profiles of NRP2, associated ligands, and receptors in melanoma cell lines from varying stages of progression using Genechip data. FIG. 5B shows the expression of VEGFR1, VEGFR2, and VEGFR3 in melanoma cell lines from varying stages of progression using quantitative RT-PCR. FIG. 5C shows the western blot results of NRP2 expression in melanoma cell lines from varying stages of progression.

FIG. 6. Cell-cell communications with different tumor cell types induces variable degree of HUVECs patterning. FIGS. 6A-6C show the patterning results of RFP-HUVECs co-cultured with different tumor cell lines following 48 hours of co-culture (top panel) and evaluated using the morphological analysis of HUVEC network formation (lower panel) for RFP-HUVECs alone (FIG. 6A), RFP-HUVEC's co-cultured with HCT-116 colon cancer cells (FIG. 6B), and RFP-HUVECs co-cultured with GFP-1205Lu melanoma cells (FIG. 6C). FIG. 6D shows the quantification of the morphological analysis of HUVEC patterning induced by co-culture with various tumor cell lines vs. HUVEC alone. FIG. 6E shows the results for the western blot analysis of NRP2 expression in various tumor cell lines grown in heterotypic co-culture with RFP-HUVECs. The tumor cell lines used were: glioblastoma, U87MG, melanoma, GFP-1205Lu, breast cancer, Hs578T, non-small cell lung carcinoma, H460, prostate cancer, PC-3, pancreatic cancer, Panc3.014, colon cancer, HCT-116, ovarian cancer, and ES-2.

FIG. 7. Representative staining for NRP2 in normal human tissues. FIG. 7A shows staining for NRP2 in normal kidney. FIG. 7B shows staining for NRP2 in striated muscle. FIG. 7C shows staining for NRP2 in testis.

FIG. 8. Representative staining for NRP2 in non-melanocytic tumors. FIG. 8A shows staining for NRP2 in colon adenocarcinoma. FIG. 8B shows staining for NRP2 in renal cell carcinoma. FIG. 8C shows staining for NRP2 in ductal breast carcinoma. FIG. 8D shows box plots demonstrating quantified staining of non-melanocytic tumor tissues (MFH-malignant fibrous histiocytoma; NSCL sqcc-non small cell lung cancer, squamous cell; RCC-renal cell carcinoma; TCC-transitional cell carcinoma/bladder).

FIG. 9. Representative staining for NRP2 in melanocytic tumors. FIG. 9A shows staining for NRP2 in metastatic amelanotic epithelioid melanoma. FIG. 9B shows staining for NRP2 in malignant melanoma. FIG. 9C shows staining for NRP2 in metastatic amelanotic spindle cell malignant melanoma. FIG. 9D shows staining for NRP2 in pigmented epithelioid melanoma. FIG. 9E shows staining for NRP2 in spindle cell nodular melanoma. FIG. 9F shows staining for NRP2 in desmoplastic malignant melanoma. FIG. 9G shows box plots demonstrating quantified staining of melanocytic tumor tissues.

FIG. 10. Quantified tissue staining for NRP2. FIG. 10 is a graphic depiction of quantified tissue staining for NRP2 in melanocytic tumors (green) and non-melanocytic tumors (pink). Values reported are the mean percent staining in tumors and melanomas positive for NRP2. Melanoma tumor results are shown in green and non-melanocytic tumor results are shown in pink.

FIG. 11. NRP2 is expressed in suprabasal keratinocytes, but not in benign nevi. FIGS. 11A-11D are low-power (10×) images of NRP2 staining in benign nevi. Note suprabasal expression of NRP2 (red) in the epidermis without staining of normal melanocytes. FIGS. 11E-11F are higher-power (20×) views of NRP2-stained benign nevi. FIG. 11F is a high-power view (40×) of NRP2-stained benign nevus.

FIG. 12. NRP2 expression is limited to metastatic melanoma cells. FIGS. 12A and 12C are low-power (FIG. 12A) and high-power (FIG. 12C) images of Melan-A stained metastatic melanoma cells within a lymph node. FIGS. 12B and 12D are low-power (FIG. 12B) and high-power (FIG. 12D) images of NRP2 stained metastatic melanoma cells within a lymph node. Arrows point to matched staining of tumor tissue by Melan-A and NRP2.

FIG. 13. NRP2 is expressed as a secreted protein. FIG. 13A shows the results for the IP-western analysis of NRP2 expression in conditioned medium from RFP-HUVECs, GFP-1205Lu melanoma cells, and HUVEC-1205Lu co-cultures. H460 (NRP1+/NRP2−) lung cancer cells were included as a negative control for NRP2. FIG. 13B shows the result of an IP-western analysis of NRP2 expression from conditioned media collected from mock and NRP2 transfected HEK293T cells, RFP-HUVECs, GFP-1205Lu melanoma cells, HUVEC-1205Lu co-cultures, and H460 (NRP1+/NRP2−) lung cancer cells.

FIG. 14. NRP2 ELISA. FIG. 14 shows the results of an ELISA assay performed on recombinant human NRP2 using the C-9 and H-300 antibodies from Santa Cruz™ (Santa Cruz, Calif.).

FIG. 15. In vivo NRP2 imaging. FIG. 15A are in vivo imaging results taken 4 hours post injection. FIG. 15B are in vivo imaging results taken 72 hours post injection. FIG. 15C are in vivo imaging results taken 120 hours post injection.

FIG. 16. Detection of Melanoma Cells Using NRP2 FACS. FIG. 16A shows the FACS results of vertical growth phase melanoma cell lines labeled and identified by extracellular staining. FIG. 16B shows the FACS results of vertical growth phase melanoma cell lines labeled and identified by intracellular staining.

### DESCRIPTION

The present invention is directed to methods for detecting and treating melanoma tumor cells. Neuropilin-2 (NRP2) is a cell surface receptor, and the present inventors have demonstrated that NRP2 is a critical mediator of melanoma cell proliferation. As such, NRP2 is a novel therapeutic target for treating melanoma. The present inventors have also demonstrated that NRP2 is present i) on melanoma tumor cells, and ii) as a soluble protein in the sera of melanoma patients. As such, NRP2 is a novel biomarker for the detection of melanoma tumor cells.

Accordingly, in some embodiments of the present invention, it is desirable to use an agent that inhibits NRP2 mediated cell proliferation to treat a subject suffering from or at risk for developing melanoma. In some embodiments, the agent is a molecule that selectively binds to NRP2. In some embodiments, the agent is an antibody that specifically binds to NRP2. The antibody can be monoclonal or polyclonal. In some embodiments, the agent is a protein that selectively binds to NRP2. In some embodiments, the protein is a VEGF or a fragment of VEGF. In other embodiments, the protein is a PLEXIN or a fragment of a PLEXIN. In other embodiments, the protein is a semaphorin or a fragment of a semaphorin. In some embodiments, the agent is detected using a conventional detection method(s) that is well-known in the art. In some embodiments, the agent is labeled. The label can be a fluorescent moiety, a moiety that binds a reporter ion, a magnetic particle, a heavy ion, a gold particle, a quantum dot, or any conventional label that is well-known in the art.

One aspect of the present invention is to use NRP2 as a biomarker i) for detecting or diagnosing melanoma in a subject, ii) to identify a subject at risk of developing melanoma, and/or iii) to predict the recurrence of melanoma in a subject. In embodiments, a sample is obtained from the subject and the biomarker is detected using a conventional detection method(s) that is well-known in the art. In some embodiments, the biological sample is tissue, a tissue homogenate, a tissue slice, a cell, a necropsy sample, a pathology sample, a biopsy sample, or bodily fluid. In some embodiments, the sample is blood, plasma, serum, urine, effusion, or spinal fluid. In embodiments, the above-described inventions further comprise identifying the presence of i) cells expressing NRP2, ii) soluble NRP2, or iii) a soluble fragment of NRP2 in the biological sample.

In some embodiments, the biomarker is identified by contacting the biological sample with an agent that selectively detects cells expressing NRP2. In some embodiments, the agent is a molecule that selectively binds to NRP2. In some embodiments, the agent is an antibody that specifically binds to NRP2. The antibody can be monoclonal or polyclonal. In some embodiments, the agent is a protein that selectively binds to NRP2. In some embodiments, the protein is a VEGF or a fragment of VEGF. In other embodiments, the protein is a PLEXIN or a fragment of a PLEXIN. In other embodiments, the protein is a semaphorin or a fragment of a semaphorin. In embodiments, the agent is detected using a conventional detection method(s) that is well-known in the art. In some embodiments, the biomarker is identified by an immunoassay, flow cytometry, affinity column separation, or magnetic selection. In some embodiments, the biomarker is identified by ELISA or immunohistochemistry. In other embodiments, the biomarker is identified by fluorescent-activated cell sorting (FACS). In embodiments, the agent is labeled. The label can be a fluorescent moiety, a moiety that binds a reporter ion, a magnetic particle, a heavy ion, a gold particle, a quantum dot, or any conventional label that is well-known in the art.

Another aspect of the present invention is to identify the presence of NRP2 melanoma cells in vivo. In embodiments, a subject is administered a diagnostically effective amount of an agent that selectively detects NRP2. In some embodiments, the agent is a molecule that selectively binds to NRP2. In some embodiments, the agent is an antibody that specifically binds to NRP2. The antibody can be monoclonal or polyclonal. In embodiments, the agent is detected using a conventional detection method(s) that is well-known in the art. In some embodiments, the agent is labeled. The label can be a fluorescent moiety; a moiety that binds a reporter ion; a magnetic particle; a heavy ion; a gold particle; a quantum dot; a radioisotope, including fluorine, iodine, bromine, and astatine; or any conventional label that is well-known in the art.

Another aspect of the present invention is directed to a method for isolating a melanoma cell from a sample. The method involves providing a sample and contacting the sample with an agent that selectively binds cells expressing neuropilin-2. In embodiments, a sample is obtained from the subject. The sample can be any bodily fluid containing cells, including blood. In some embodiments, the agent is an antibody that specifically binds to NRP2. The antibody can be monoclonal or polyclonal. In some embodiments, the agent is a protein that selectively binds to NRP2. In some embodiments, the protein is a VEGF or a fragment of VEGF. In other embodiments, the protein is a PLEXIN or a fragment of a PLEXIN. In other embodiments, the protein is a semaphorin or a fragment of a semaphorin. In embodiments, the agent is detected using a conventional detection method(s) that is well-known in the art. In some embodiments, the agent is labeled. The label can be a fluorescent moiety, a moiety that binds a reporter ion, a magnetic particle, a heavy ion, a gold particle, a quantum dot, or any conventional label that is well-known in the art. In embodiments, the method further comprises washing the isolated cell(s) with an aqueous medium. In some embodiments, the method further comprises conducting molecular analysis of the isolated cell(s). Molecular analysis includes any conventional molecular assay that is well-known in the art, including genetic analysis of the isolated cell(s). In embodiments, the results of the molecular analysis are used to guide patient treatment strategies.

A further aspect of the present invention is a kit for i) detecting melanoma in a subject, ii) identifying a subject at risk for developing melanoma, or iii) predicting recurrence of melanoma in a subject. In embodiments, the kit contains an agent that detects the presence of i) cells expressing NRP2, ii) soluble NRP2, or iii) a soluble fragment of NRP2. In some embodiments, the agent is an antibody that specifically binds to NRP2 or a soluble fragment of NRP2. The antibody can be monoclonal or polyclonal. In some embodiments, the agent is a protein that selectively binds to NRP2. In some embodiments, the protein is a VEGF or a fragment of VEGF. In other embodiments, the protein is a PLEXIN or a fragment of a PLEXIN. In other embodiments, the protein is a semaphorin or a fragment of a semaphorin. In embodiments, the agent is detected using a conventional detection method(s) that is well-known in the art. In some embodiments, the agent is labeled. The label can be a fluorescent moiety, a moiety that binds a reporter ion, a magnetic particle, a heavy ion, a gold particle, a quantum dot, or any conventional label that is well-known in the art.

To facilitate an understanding of the present invention, a number of terms and phrases are defined below.

As used herein, the singular forms “a”, “an”, and “the” include plural forms unless the context clearly dictates otherwise. Thus, for example, reference to “a protein” includes reference to more than one protein.

As used herein, “neuropilin-2” and “NRP2” include the full length, isomers, and fragments of the transmembrane glycoprotein. Also included within the definition are neuropilin-2 that have been modified naturally or by intervention, for example, by disulfide bond formation, glycosylation, lipidation, acetylation, phosphorylation, or any other manipulation or modification, such as conjugation with a labeling component. Further included within the definition are, for example, neuropilin-2 containing one or more analogs of an amino acid (including, for example, unnatural amino acids, etc.), as well as other modifications that are conventional and well-known in the art.

As used herein, “melanoma” includes, but is not limited to, a growth of malignant melanocytes, primary melanomas, metastatic melanomas, melanomas derived from either melanocytes or melanocytes related nevus cells, melanocarcinomas, melanoepitheliomas, melanosarcomas, melanoma in situ, superficial spreading melanoma, nodular melanoma, lentigo maligna melanoma, acral lentiginous melanoma, invasive melanoma or familial atypical mole and melanoma (FAM-M) syndrome. Such melanomas in mammals may be caused by, chromosomal abnormalities, degenerative growth and developmental disorders, mitogenic agents, ultraviolet radiation (UV), viral infections, inappropriate tissue expression of a gene, alterations in expression of a gene, or carcinogenic agents. The aforementioned melanomas can be diagnosed, assessed or treated by methods described in the present application.

A sample which is “provided” can be obtained by the person (or machine) conducting the assay, or it can have been obtained by another, and transferred to the person (or machine) carrying out the assay.

By a “sample” (e.g. a test sample) from a subject is meant a sample that might be expected to contain elevated levels of the protein markers of the invention in a subject having heart failure. Many suitable sample types will be evident to a skilled worker. In some embodiments, the sample is a blood sample, such as whole blood, plasma, or serum (plasma from which clotting factors have been removed). For example, peripheral, arterial or venous plasma or serum can be used. In some embodiments, the sample is urine, sweat, or another body fluid, such as spinal fluid, into which proteins are sometimes removed from the blood stream. In the case of urine, for example, the protein is likely to be broken down, so diagnostic fragments of the proteins of the invention can be screened for. In some embodiments, the sample is tissue, including tissue slices, tissue homogenates, or primary cultures of mammalian tissues; a cell, a necropsy sample, a pathology sample, a biopsy sample. Methods for obtaining samples and preparing them for analysis (e.g., for detection of the amount of protein) are conventional and are well-known in the art.

A “subject,” as used herein, includes any animal that has, or is at risk of developing, melanoma. Suitable subjects (patients) include laboratory animals (such as mouse, rat, rabbit, guinea pig or pig), farm animals, sporting animals (e.g., dogs or horses), domestic animals, and pets (such as a horse, dog or cat). Non-human primates and human patients are included. In addition, subjects not exhibiting these symptoms can also be evaluated by a method of the present invention.

“At risk of” is intended to mean at increased risk of, compared to a normal subject, or compared to a control group, e.g., a patient population. Thus, a subject carrying a particular marker may have an increased risk for a specific disease or disorder, and be identified as needing further testing. “Increased risk” or “elevated risk” mean any statistically significant increase in the probability, e.g., that the subject has the disorder.

Although much of the data presented in the Examples herein are directed to full length forms of NRP2, it will be evident to a skilled worker that a variety of forms of this protein may be indicative of a subject having melanoma or the risk of developing melanoma in a subject. For example, the protein may be an intact, full-length NRP-2. In addition, the protein may be degraded and/or fragmented forms of NRP2. In such a case, an investigator can determine the level of one or more of the fragments or degradation products. Furthermore, when NRP2 undergoes processing naturally (e.g., posttranslational modifications, such as acetylation, methylation, phosphorylation, etc.), any of these forms of the protein are included in the invention. As such, “neuropilin-2” or “NRP2” refer to full-length NRP2, a fragment of NRP2, and posttranslationally modified forms of NRP2.

In some embodiments, it is desirable to express the results of an assay in terms of an increase (e.g., a statistically significant increase) in a value (or combination of values) compared to a baseline value.

A “significant” increase in a value, as used herein, can refer to a difference which is reproducible or statistically significant, as determined using statistical methods that are appropriate and well-known in the art, generally with a probability value of less than five percent chance of the change being due to random variation. Suitable statistical tests will be evident to a person of ordinary skill in the art. A significantly elevated amount of a protein of the invention compared to a suitable baseline value, then, is indicative that a test subject has melanoma or is at risk of developing melanoma. A subject is “likely” to have or be at risk for developing melanoma if the subject has levels of the marker protein significantly above those of a healthy control or his own baseline (taken at an earlier time point). The extent of the increased levels correlates to the % chance. In general, the presence of an elevated amount of a marker of the invention is a strong indication that the subject has melanoma.

As used herein, a “baseline value” generally refers to the level (amount) of a protein in a comparable sample (e.g., from the same type of tissue as the tested tissue), from a “normal” healthy subject that does not have melanoma. If desired, a pool or population of the same tissues from normal subjects can be used, and the baseline value can be an average or mean of the measurements. Suitable baseline values can be determined by those of skill in the art without undue experimentation. Suitable baseline values may be available in a database compiled from the values and/or may be determined based on published data or on retrospective studies of patients' tissues, and other information as would be apparent to a person of ordinary skill implementing a method of the invention. Suitable baseline values may be selected using statistical tools that provide an appropriate confidence interval so that measured levels that fall outside the standard value can be accepted as being aberrant from a diagnostic perspective, and predictive of melanoma.

It is generally not practical in a clinical or research setting to use patient samples as sources for baseline controls. Therefore, one can use any of variety of reference values in which the same or a similar level of expression is found in a subject that does not have melanoma.

It will be appreciated by a person of ordinary skill in the art that a baseline or normal level need not be established for each assay as the assay is performed, but rather, baseline or normal levels can be established by referring to a form of stored information regarding a previously determined baseline levels for a given protein or panel of proteins, such as a baseline level established by using any of the methods described herein. Such a form of stored information can include, for example, a reference chart, listing or electronic file of population or individual data regarding “normal levels” (negative control) or positive controls; a medical chart for the patient recording data from previous evaluations; a receiver-operator characteristic (ROC) curve; or any other source of data regarding baseline levels that is useful for the patient to be diagnosed. In some embodiments the amount of the proteins in a combination of proteins, compared to a baseline value, is expressed as a linear regression score, as described, e.g., in Irwin, in Neter, Kutner, Nachtsteim, Wasserman (1996) Applied Linear Statistical Models, 4^{th} edition, page 295.

In some embodiments in which the progress of a treatment is being monitored, a baseline value can be based on earlier measurements taken from the same subject, before the treatment was administered.

The amount of a protein can be measured using any suitable method. Some methods involve the use of antibodies, binding ligands, or mass spectrometry tagged peptides specific for a protein of interest. Antibodies suitable for use in assays of the invention are commercially available, or can be prepared routinely. Methods for preparing and using antibodies in assays for proteins of interest are conventional, and are described, e.g., in Green et al., Production of Polyclonal Antisera, in Immunochemical Protocols, Manson ed. (Humana Press 1992); Coligan et al., in Current Protocols in Immunology, sections 2.4.1 and 2.5.1-2.6.7 (1992); Köhler & Milstein, Nature 256:495-7 (1975); and Harlow et al., Antibodies: A Laboratory Manual, page 726 (Cold Spring Harbor Laboratory Pub. 1988).

Immortalized human B lymphocytes immunized in vitro or isolated from an immunized individual that produce an antibody directed against a target antigen can be generated. See, e.g., Cole et al., Monoclonal Antibodies and Cancer Therapy, Alan R. Liss ed., p. 77 (1985); Boerner et al., J Immunol, 147 (1):86-95 (1991); and U.S. Pat. No. 5,750,373.

Also, the human antibody can be selected from a phage library, where that phage library expresses human antibodies, as described, for example, in Vaughan et al., Na. Biotech, 14:309-314 (1996), Sheets et al., Proc Natl Acad Sci, 95:6157-6162 (1998), Hoogenboom and Winter, 1991, J. Mol. Biol., 227:381, and Marks et al., J Mol Biol, 222:581 (1991). Techniques for the generation and use of antibody phage libraries are also described in U.S. Pat. Nos. 5,969,108, 6,172,197, 5,885,793; 6,521,404; 6,544,731; 6,555,313; 6,582,915; 6,593,081; 6,300,064; 6,653,068; 6,706,484; and 7,264,963; and Rothe et al., J Mol Bio, J Mol Biol 376:1182-1200 (2007). Affinity maturation strategies, such as chain shuffling (Marks et al., Bio/Technology 10:779-783 (1992)), are known in the art and may be employed to generate high affinity human antibodies.

Humanized antibodies can also be made in transgenic mice containing human immunoglobulin loci that are capable upon immunization of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

Any of a variety of antibodies can be used in methods of the invention. Such antibodies include, e.g., polyclonal, monoclonal (mAbs), recombinant, humanized or partially humanized, single chain, Fab, and fragments thereof. The antibodies can be of any isotype, e.g., IgM, various IgG isotypes such as IgG₁, IgG₂ₐ, etc., and they can be from any animal species that produces antibodies, including goat, rabbit, mouse, chicken or the like. The term, an antibody “specific for” or that “specifically binds” a protein, means that the antibody recognizes a defined sequence of amino acids, or epitope in the protein. An antibody that is “specific for,” “specifically recognizes,” or that “specifically binds” a polypeptide refers to an antibody that binds selectively to the polypeptide and not generally to other polypeptides unintended for binding to the antibody. The parameters required to achieve such specificity can be determined routinely, using conventional methods in the art. Conditions that are effective for binding a protein to an antibody which is specific for it are conventional and well-known in the art.

“Detectable moiety” or a “label” refers to a composition detectable by spectroscopic, photochemical, biochemical, immunochemical, radioactive, or chemical means. For example, useful labels include ³²P, ³⁵S, fluorescent dyes, electron-dense reagents, enzymes (e.g., as commonly used in an ELISA), biotin-streptavidin, dioxigenin, haptens and proteins for which antisera or monoclonal antibodies are available, or nucleic acid molecules with a sequence complementary to a target. The detectable moiety often generates a measurable signal, such as a radioactive, chromogenic, or fluorescent signal, that can be used to quantify the amount of bound detectable moiety in a sample. Quantitation of the signal is achieved by, e.g., scintillation counting, densitometry, flow cytometry, ELISA, or direct analysis by mass spectrometry of intact or subsequently digested peptides (one or more peptide can be assessed). Persons of skill in the art are familiar with techniques for labeling compounds of interest, and means for detection. Such techniques and methods are conventional and well-known in the art.

In embodiments of the present invention, antibodies specific for a protein of the present invention are immobilized on a surface (e.g., are reactive elements on an array, such as a microarray, or are on another surface, such as used for surface plasmon resonance (SPR)-based technology, such as BIAcore), and proteins in the sample are detected by virtue of their ability to bind specifically to the antibodies. Alternatively, proteins in the sample can be immobilized on a surface, and detected by virtue of their ability to bind specifically to the antibodies. Methods of preparing the surfaces and performing the analyses, including conditions effective for specific binding, are conventional and well-known in the art.

Among the many types of suitable immunoassays are competitive and non-competitive assay systems using techniques such as BIAcore analysis, FACS analysis, immunofluorescence, immunohistochemical staining, Western blots (immunobots), radioimmunoassays, ELISA, “sandwich” immunoassays, immunoprecipitation assays, precipitation reactions, gel diffusion precipitin reactions, immunodiffusion assays, agglutination assays, complement-fixation assays, immunoradiometric assays, fluorescent immunoassays, fluorescence-activated cell sorting (FACS), protein A immunoassays, etc. Assays used in a method of the invention can be based on colorimetric readouts, fluorescent readouts, mass spectrometry, visual inspection, etc. Assays can be carried out, e.g., with suspension beads, or with arrays, in which antibodies or cell or blood samples are attached to a surface such as a glass slide or a chip.

In embodiments of the present invention, a tissue sample is stained with a suitable antibody in a conventional immunohistochemical assay(s) well-known in the art for those proteins which are present in the tissue.

In general, molecular biology methods referred to herein are well-known in the art and are described, e.g., in Sambrook et al., Molecular Cloning: A Laboratory Manual, current edition, Cold Spring Harbor Laboratory, Cold Spring Harbor, N.Y., and Ausubel et al., Current Protocols in Molecular Biology, John Wiley & Sons, New York, N.Y.

“Diagnostic” means identifying the presence or nature of a pathologic condition and includes identifying patients who are at risk of developing a specific disease or disorder. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

A detection (diagnostic) method of the invention can be adapted for many uses. For example, it can be used to follow the progression of melanoma. In embodiment of the present invention, the detection is carried out both before (or at approximately the same time as), and after, the administration of a treatment, and the method is used to monitor the effectiveness of the treatment. A subject can be monitored in this way to determine the effectiveness for that subject of a particular drug regimen, or a drug or other treatment modality can be evaluated in a pre-clinical or clinical trial. If a treatment method is successful, the levels of the protein markers of the invention are expected to decrease.

As used herein, “treated” means that an effective amount of a drug or other anti-melanoma procedure is administered to the subject. An “effective” amount of an agent refers to an amount that elicits a detectable response (e.g. of a therapeutic response) in the subject.

One aspect of the invention is a method for isolating a melanoma cell using an agent that selectively binds to NRP2. In embodiments, the present invention is employed to isolate rare cells from a sample. In some embodiments, the rare cells are circulating melanoma cells from peripheral blood. In general, devices for use in isolating rare cells are well-known in the art, e.g., Cellsearch™ System by Veridex LLC. A person of ordinary skill in the art will recognize the experimental conditions and systems that can be used to isolate single cells from a sample.

In addition to methods of isolating biological cells from a sample, some embodiments of the present invention provide methods in which the isolated cells may be used to provide additional information. In embodiments, cells isolated using the methods of the present invention can be further assayed using additional in vitro assays. In some embodiments, cells that are isolated using the methods of the present invention are counted. Conventional methods for counting cells can be used in some embodiments, including for example, optical, e.g., visual inspection, automated counting, microscopy based detection; FACS; and electrical detection, e.g., Coulter counters. Cell counting can be useful for diagnosing disease, monitoring the progress of disease, and monitoring or determining the efficacy of a treatment.

In embodiments, cells isolated using the methods of the present invention are subjected to immunocytochemical analysis by flowcytometry or other analytical platforms. Such analysis facilitates diagnosis and provides important information to the clinician.

In some embodiments, cells isolated using the methods of the present invention can be lysed, and one or more properties of the cells, or portions thereof, can be measured. Nonlimiting examples of biological properties that can be measured in lysed cells include mRNA expression, protein expression, and DNA quantification. Additionally, in some embodiments, the cellular DNA can be sequenced, or certain sequence characteristics (e.g., polymorphisms and chromosomal abnormalities) can be identified using conventional techniques, e.g., FISH or PCR. In some embodiments, cells are lysed while still bound to the device.

In some embodiments, cells isolated by the methods of the present invention are assayed without lysis. Nonlimiting examples of methods for assaying non-lysed cells include using extracellular or intracellular stains; observing morphology or growth characteristics in various media; and identifying biomarkers on the cellular surface. In further embodiments, the isolated cells are cultured to obtain an enriched population of the isolated cells before use in subsequent in vitro assays.

In some embodiments of the present invention, information that can be obtained from the isolated cells includes identification or enumeration of particular genomic DNA, cDNA, or mRNA sequences; identification or enumeration of cell surface markers; and identification or enumeration of proteins or other intracellular contents that are indicative of the type or presence of a particular tumor. In embodiments, isolated cells may be analyzed to determine the tissue of origin, the stage or severity of disease, or susceptibility to a particular treatment.

In some embodiments, the methods of the present invention are used to assess residual melanoma cells in circulation following medical, radiation, or surgical treatment to eradicate the melanoma tumor. In further embodiments, the methods and devices of the present invention are performed periodically over a course of years to assess the patient for the presence and number of melanoma cells in the circulation as an indicator of occurrence, recurrence and/or progression of disease.

Another aspect of the invention is a kit for detecting whether a subject is suffering from or at risk for developing melanoma, comprising one or more agents for detecting the amount of a protein of the invention. The kit may also include additional agents suitable for detecting, measuring and/or quantitating the amount of protein, including conventional analytes for creation of standard curves. Among other uses, kits of the invention can be used in experimental applications. A person of ordinary skill in the art will recognize components of kits suitable for carrying out a method of the present invention.

If an antibody-based method is to be used to measure protein levels, the agents in the kit can encompass antibodies specific for the proteins. In some embodiments, the antibodies are labeled with a detectable marker, e.g., a chemiluminescent, enzymatic, fluorescent, or radioactive moiety. In some embodiments, the kit includes a labeled binding partner(s) to the antibodies. Antibody-based kits for protein detection are conventional and well-known in the art. A person of ordinary skill in the art will recognize components of kits suitable for detecting a biomarker(s) using antibodies.

In some embodiments, the kit contains a protein that is a binding partner of NRP2. The NRP2 binding partners can be labeled with a detectable marker, e.g., a chemiluminescent, enzymatic, fluorescent, or radioactive moiety. The kit can also include a labeled binding partner(s) to the protein that selectively binds NRP2. Such kits are well-known in the art and a person of ordinary skill in the art will recognize components of kits suitable for detecting a biomarker(s) using an NRP2 binding partner as the binding agent.

In embodiments, a kit of the invention may comprise instructions for performing the method. Optionally, the kit can include instructions for taking a sample from the mammalian subject (e.g., body fluid), and using the kit to identify a mammalian subject suffering from or at risk of developing melanoma. In some embodiments, a kit of the invention contains suitable buffers, containers, or packaging materials. The reagents of the kit may be in containers in which the reagents are stable, e.g., in lyophilized form or stabilized liquids. The reagents may also be in single use form, e.g., for the performance of an assay for a single subject.

Embodiments of the present invention can be further defined by reference to the following non-limiting examples, which describe the methodology employed to identify and characterize NRP2 as a novel biomarker and therapeutic target for melanoma. It will be apparent to those skilled in the art that many modifications, both to materials and methods, may be practiced without departing from the scope of the present disclosure.

### EXAMPLES

Neuropilin-2 has been identified as a novel biomarker of melanoma tumor cells. It is understood that the examples and embodiments described herein are for illustrative purposes only and that various modifications or changes in light thereof will be suggested to persons skilled in the art and are to be included within the spirit and purview of this application.

### Example I

### Melanoma-Endothelial Cell Communication is Observed In Vitro

Tumor cell interactions with neighboring endothelial cells are critical for tumor survival and metastasis. Melanomas are notorious for their ability to metastasize at a relatively early stage of development. This aggressive behavior depends, at least in part, on the interaction between tumor cells and their surrounding stroma. Using a long-term heterotypic cell co-culture system to model tumor-stromal cellular interactions that occur during metastasis in vivo, neuropilin-2 (NRP2), a cell surface receptor involved in angiogenesis and axonal guidance, has been identified as a gene that is highly upregulated in melanoma cells during melanoma and endothelial cells interactions.

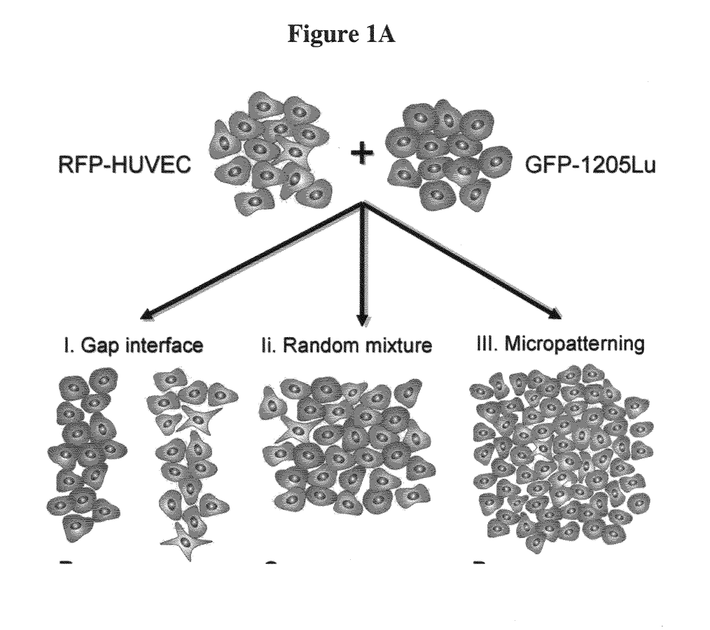

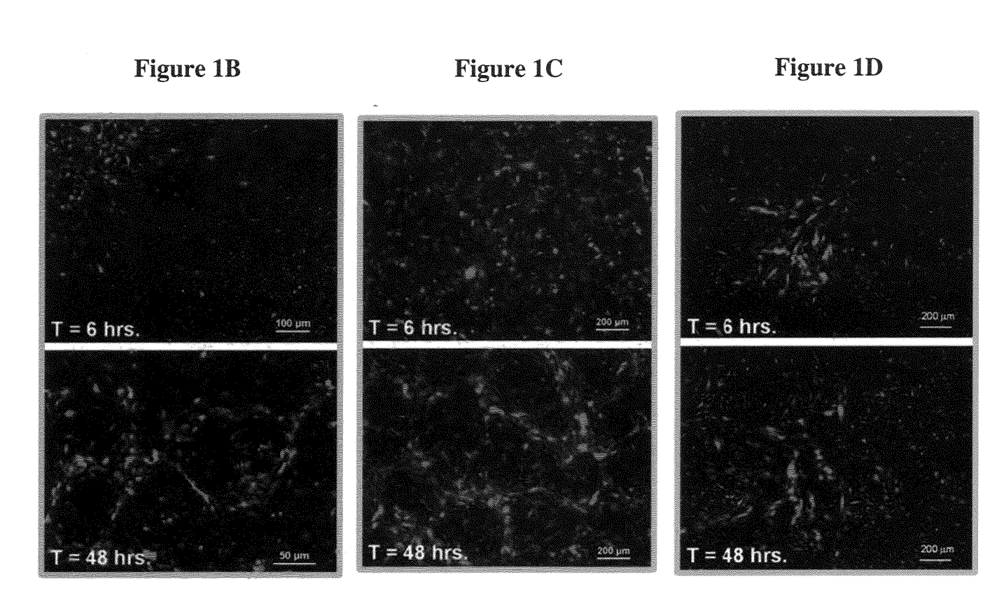

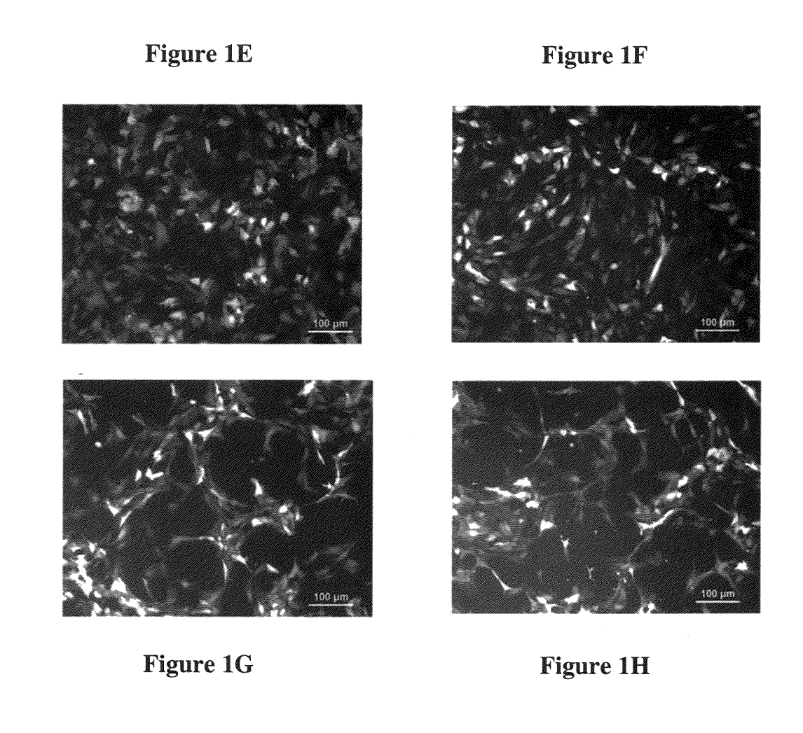

Three distinct in vitro two-dimensional co-culture systems of melanoma and endothelial cells were utilized to identify the molecular determinants of melanoma-endothelial cell communication (FIG. 1A). These systems were designed to increase the degree of control of the co-culture and to easily distinguish constituent cell lines. Cell lines used in these studies include the metastatic melanoma cell line, 1205Lu, carrying stably-integrated green fluorescent protein (GFP-1205Lu) and human umbilical vein endothelial cells stably transfected with red fluorescent protein (RFP-HUVECs).

In the first system (the defined gap method), separate adjacent colonies of melanoma and endothelial cells were incubated in such a way that initially the colonies were separated by 150-200 pm (FIG. 1A, left). 1.5×10⁵ RFP-HUVECs (human umbilical vein endothelial cells) were seeded in EGM-2 inside a plastic cloning cylinder (10 mm inner diameter) in a 24-well plate. 3×10⁵ GFP-1205Lu cells were plated outside the cloning ring. After a 4 hour incubation to allow the cells to adhere to the plate, the cloning ring was removed, leaving a 150-200 pm gap between the heterotypic cell populations. The cells were washed with PBS, and fresh EGM-2 was added to the wells. The cells were then allowed to migrate for 24 hours, after which photographs were taken of the wells. After 48 hrs, the heterotypic cells interacted and created a self-organizing cellular networks at the interface.

In the second system (the random mixture method), cells were pre-mixed at a 50/50 ratio, plated in this randomly mixed state and allowed to interact for the subsequent 48 hrs (FIG. 1A, middle). GFP-1205Lu and RFP-HUVEC were plated at 95% confluency at a 1:1 ratio in EGM-2 culture medium. Cells were incubated for 48 hours before being sorted into pure populations, using fluorescence activated cell sorting (FACS). Control cultures of individual cell types were bgfregrown under identical conditions. Co-cultured and control cells were washed 2× with PBS and collected by trypsinization. Collected cells were resuspended for FACS in ice-cold EGM-2.

The third system (the micropatterning method) employs stencil-based technology. Endothelial cells were patterned into circular or triangular shaped colonies of pre-defined size, with melanoma cells then plated into the remaining surrounding open spaces (FIG. 1A, right). Specifically, RFP-HUVECs were micropatterned into two-dimensional colonies using a microstencil technique similar to that reported by Ostuni et al [1]. SU-8, a high-aspect ratio negative photoresist, was photopatterned onto silicon. This was used as a mold for spin-casting 100 pm tall polydimethylsiloxane (PDMS) membrane. The elastomeric membrane was laid flat on a collagen coated glass substrate surface, then exposed to culture medium that contained HUVEC cells. Cells adhered to both the PDMS stencil and the glass substrate. Upon peeling of the membrane, HUVECs remained in the area not protected by the microstencil.

The number of cells in each of the circular and triangular shaped colony was controlled precisely (88+/−14 cells for 1 mm diameter circular colony; 72+/−20 cells for triangular colony with 1 mm sides). To distinguish melanoma and endothelial cells during and following co-incubation, the metastatic melanoma cell line, 1205Lu, carrying stably-integrated green fluorescent protein (GFP-1205Lu) and human umbilical vein endothelial cells stably transfected with red fluorescent protein (RFP-HUVECs) were used. The three different co-incubation techniques employed primarily differ in terms of the nature and the extent of the interface between heterotypic cells: the interface is initially absent but is emerging in the first method, it is very extensive but poorly controlled in the second method, and it is well defined and controlled but not very extensive in the third method. These differentially defined interfaces allow the user to vary the extent of heterotypic cell interactions that might preferentially occur as a result of modulating their spatial relationship.

The changes in the positions of individual HUVEC cells was manually tracked over time and the coordinates were recorded using custom-made software package implemented in Mathematica (version 6). The spatial correlation factor of the velocity vectors was calculated as a function of cell-cell distance r (not exceeding 200 μM in our analysis) for all cell pairs, as described previously [2]:

$$C ( r ) = {\sum }_{i , j}^{r = \vert {r}_{o} - {r}_{j} \vert} \frac{( {\vec{v}}_{i} \cdot {\vec{v}}_{j} )}{\vert {\vec{v}}_{i} \vert \vert {\vec{v}}_{j} \vert}$$

where v, denotes the velocity vector of cell i, at position r, calculated from the difference between the cell positions in two images taken at an interval of 1 hour. When movement of a pair of cells is highly correlated, the value of C(r) can approach 1. Lower values of C(r) indicate lower correlation in the directional movement.

In the defined gap method, striking endothelial cell networks developed as the tumor-endothelial cell interface gradually emerged and cells progressively intermingled (FIG. 1B, bottom). Notably, these structures included continuous loops composed of endothelial cells that enclosed tumor cells and were of shape and form analogous to those seen in tumor parenchyma [3, 4]. This structured assembly of endothelial cells differed from the random assembly observed with endothelial cells cultured alone (FIG. 1F).

Evaluation of random co-cultures of melanoma and endothelial cells, using method the random mixture, showed minimal HUVEC network formation at 6 hours of co-culture (FIG. 1C, top), but pronounced cell network formation at 48 hours (FIG. 1C, bottom) of co-culture. The networks formed by a gradual accumulation of endothelial cells into the looped and branched structures clearly distinguishable against the background of melanoma cells, with cell network morphology similar to that observed using the defined gap method.

Finally, in the micropatterning method, it was found that, over the 48 hrs. of co-incubation, endothelial cells moved in collective migratory “streams” out of the pre-patterned islands into the surrounding large scale colonies of melanoma cells, forming the initial branched network (FIG. 1D). Thus all three cell co-incubation methods resulted in an extensive reorganization of endothelial cells into branched and looped networks at the interfaces between heterotypic cell colonies.

Incubation of HUVECs in either basal medium alone or HUVEC-conditioned media for 48 hours failed to elicit HUVEC patterning in vitro (FIGS. 1E and 1F). However, in HUVECs incubated with either melanoma cell-conditioned media or co-culture-conditioned media, cells reorganized into networks similar to those found using the defined gap and random mixture co-culture methods at 48 hours of incubation (FIGS. 1G and 1H), suggesting that melanoma-HUVEC communication is mediated, at least in part, by one or more tumor-secreted soluble factor(s).

### Example II

### Differential Gene Expression During Melanoma-Endothelial Cell Communication

Among the co-culture methods used, the random mixture method is particularly advantageous for a large-scale evaluation in changes of genomic composition of cells participating in co-culture due to similar relative numbers of and an extensive interface between heterotypic cells, making it likely that most cells would take part in cell-cell endothelial-melanoma cell-cell communication. Therefore, using the random mixture method followed by sorting of heterotypic cells based on the color of the cell type-specific fluorescent labels, gene expression profiles of the sorted cells were examined.

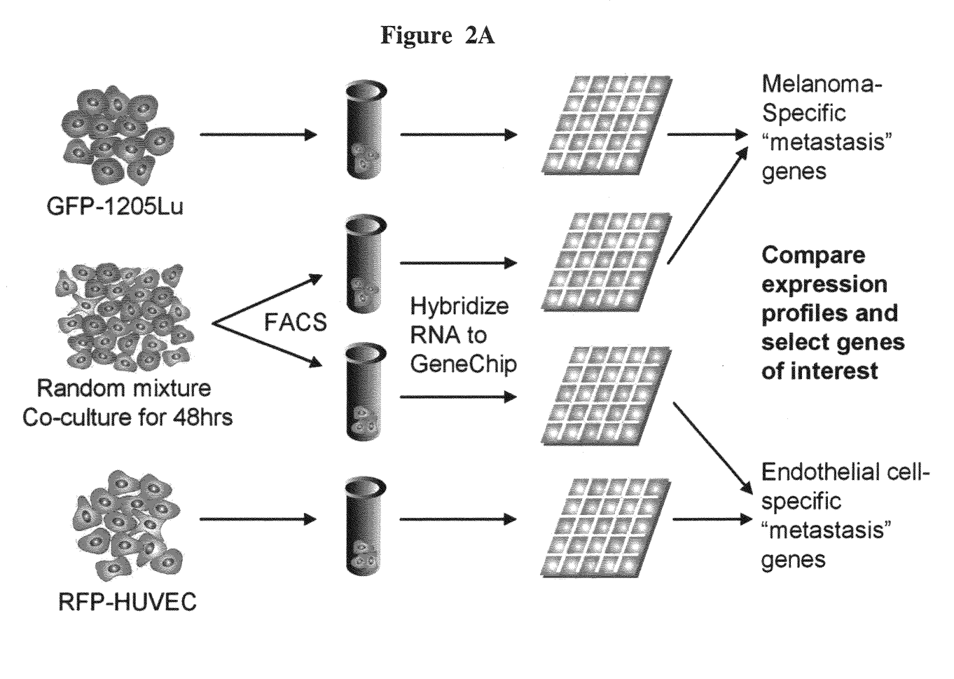

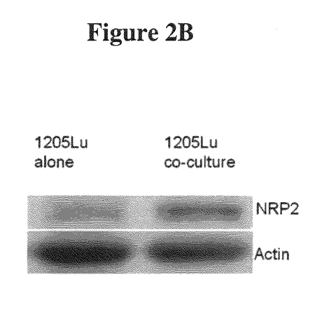

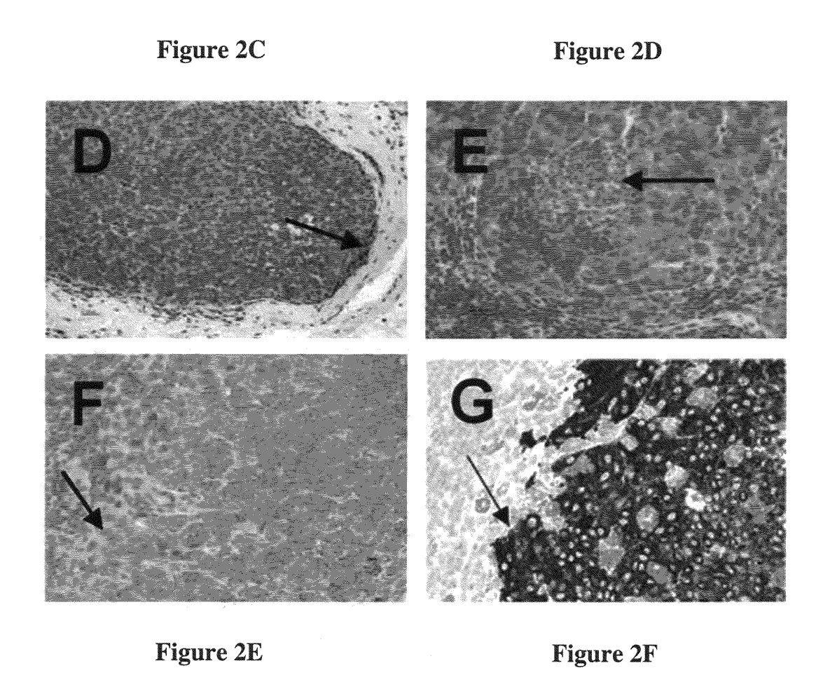

To define the molecular pathways governing melanoma-HUVEC communication, gene expression profiling of GFP-1205Lu and RFP-HUVECs cultured alone or in mixture for 48 hrs (FIG. 2A) were performed. Microarray analysis was performed as previously described [5]. A spreadsheet containing the expression levels from the four GeneChips (GFP-1205Lu alone, GFP-1205Lu co-culture, RFP-HUVEC alone, RFP-HUVEC co-culture) was generated in Microsoft Excel. The signals from the chips were normalized using RMAExpress. Two new spreadsheets were produced to generate a ratio of the expression between the co-cultured and control samples for each cell type. Only the genes tagged as present (“P”) by the Affymetrix software were considered for evaluation. A cutoff signal ratio of 2 and above was considered upregulated in co-cultured cells, and a signal of 0.5 and below was considered downregulated. Spotfire (Tibco, Somerville, Mass.) was used for further analysis of microarray data, including annotation of genes of interest with their Gene Ontology (GO) classifications. Both the classifications associated with specific genes of interest and the most common classifications among all the altered genes were assessed. Selected GO classifications associated with genes altered in co-cultured vs. monocultured GFP-1205Lu, with a p-value of less than 0.05 are shown in Table 1. All samples were run in commercial arrays from Affymetrix, using Affymetrix GeneChip human U133Plus 2.0 arrays as described in the Affymetrix web site. The JHMI Microarray Core Facility abides in all its procedures by current MIAME guidelines. Microarray data has been submitted to the Gene Expression Omnibus (GEO) repository under the series record GSE8699.

TABLE 1
Melanoma-endothelial cell co-culture promotes
the development of an invasive tumor phenotype.

| GO Biological Process | | Number of | Number | Number |
|---|---|---|---|---|
| Description | p-value | Transcripts | Upregulated | Downregulated |
| Cell adhesion | 8.94e−07 | 55 | 45 | 10 |
| Regulation of cell-cell adhesion | 0.015 | 4 | 4 | 0 |
| Cell differentiation | 0.0288 | 57 | 45 | 12 |
| Negative regulation of cell | 0.0214 | 9 | 8 | 1 |
| differentiation | | | | |
| DNA replication initiation | 0.000971 | 6 | 0 | 6 |
| Regulation of apoptosis | 0.000703 | 33 | 22 | 11 |
| Angiogenesis | 0.00111 | 21 | 19 | 2 |
| Regulation of cell migration | 0.00744 | 12 | 12 | 0 |
| Cell proliferation | 0.000397 | 52 | 26 | 26 |
| ECM organization | 0.000268 | 9 | 8 | 1 |
| Intercellular junction | 0.0436 | 10 | 10 | 0 |

Gene Ontology Biological Process descriptions associated with transcripts altered in melanoma cells following co-culture with HUVECs. Significant alterations are seen in genes associated with an aggressive tumor phenotype including altered cell migration, adhesion, and angiogenesis.

TABLE 2
Gene expression profiling data for the top 30 genes upregulated
in melanoma cells following co-culture with HUVECs.

| Probeset ID | Gene Title | Gene Symbol | Fold |
|---|---|---|---|
| 216438_s_at | thymosin, beta 4, X-linked | TMSB4X | 36.67 |
| 205612_at | multimerin 1 | MMRN1 | 14.93 |
| 201859_at | proteoglycan 1, secretory granule | PRG1 | 14.79 |
| 214841_at | cornichon homolog 3 (Drosophila) | CNIH3 | 9.13 |
| 1556499_s_at | collagen, type 1, alpha 1 | COL1A1 | 8.94 |
| 1555623_at | DERP12 (dermal papilla derived protein 12) | DERP12 | 7.80 |
| 232113_at | Hypothetical gene supported by BX647608 | — | 7.16 |
| 225566_at | neuropilin 2 | NRP2 | 6.33 |
| 823_at | chemokine (C-X3-C motif) ligand 1 | CX3CL1 | 6.31 |
| 226158_at | kelch-like 24 (Drosophila) | KLHL24 | 6.21 |
| 207147_at | distal-less homeo box 2 | DLX2 | 5.95 |
| 230538_at | rai-like protein | RaLP | 5.73 |
| 212706_at | RAS p21 protein activator 4 | RASA4 | 5.71 |
| 201667_at | gap junction protein, alpha 1, 43 kDa (connexin 43) | GJA1 | 5.65 |
| 201438_at | collagen, type VI, alpha 3 | COL6A3 | 5.61 |
| 237169_at | Tenascin C (hexabrachion) | TNC | 5.49 |
| 227020_at | yippee-like 2 (Drosophila) | YPEL2 | 5.47 |
| 213413_at | stoned B-like factor | SBLF | 5.46 |
| 203238_s_at | Notch homolog 3 (Drosophila) | NOTCH3 | 5.42 |
| 201858_s_at | proteoglycan 1, secretory granule | PRG1 | 5.10 |
| 209071_s_at | regulator of G-protein signalling 5 | RGS5 | 5.01 |
| 202112_at | von Willebrand factor | VWF | 4.98 |
| 238067_at | FLJ20298 protein | FLJ20298 | 4.89 |
| 225728_at | Importin 9 | IPO9 | 4.82 |
| 44783_s_at | hairy/enhancer-of-split related with YRPW motif 1 | HEY1 | 4.78 |
| 229225_at | neuropilin 2 | NRP2 | 4.77 |
| 226436_at | Ras association (RalGDS/AF-6) domain family 4 | RASSF4 | 4.75 |
| 214632_at | neuropilin 2 | NRP2 | 4.72 |
| 232797_at | Integrin, alpha V | ITGAV | 4.72 |
| 231779_at | interleukin-1 receptor-associated kinase 2 | IRAK2 | 4.60 |

Analysis of gene expression signatures associated with the co-culture system demonstrated specific influences of tumor cells on endothelial cell expression profiles and vice versa. The initial analysis focused on the expression profile differences in melanoma cells. Evaluation of the Gene Ontology biological classifications of the genes upregulated in GFP-1205Lu cells following co-culture with HUVECs versus melanoma cells grown alone demonstrated alterations in genes controlling cell adhesion, cell migration, extracellular matrix organization, and angiogenesis, i.e., signatures frequently associated with tumor progression and metastasis (Table 1). Although previous investigations have remarked on the vasculogenic phenotype of advanced melanomas [6-9] which have been variously attributed to cell-autonomous angiogenic properties of melanoma cells themselves, the current study suggests a more complex picture, according to which melanoma cells can be induced by neighboring endothelial cells to produce factors that can in turn influence endothelial cells, and their propensity to form functional vasculature.

Among the top 30 genes upregulated in melanoma cells under co-culture conditions were thymosin beta 4, a gene previously associated with tumor angiogenesis and melanoma metastasis [10-12], and multimerin 1, a gene involved in endothelial cell adhesion [13]. In addition, a closer examination of the highly upregulated genes suggests that many of them code for secreted components that might be responsible for ensuring fast and robust formation of de novo vascular beds. In particular, collagens types I and IV can affect the migration of both melanoma cells themselves (notably the pertinent integrin alpha V is also overexpressed in melanoma cells following co-culture) as well as ensuring enhanced attachment and migration of endothelial cells, serving as putative indirect means of cell communication [14]. Von Willebrand factor and the related protein multimerin might facilitate angiogenesis through regulation of coagulation [15]. Overexpression of cytokine CX3CL1 has been implicated in chemoattraction of several cell types, including endothelial cells derived from the skin [16]. Also, notably, NRP2, can exist in a secreted form and affect VEGF signaling on the surface of endothelial cells thus directly modulating cell signaling regulating cell proliferation and migration. These results suggest that melanoma cells can be induced by endothelial cells to produce multiple soluble factors that might influence the ensuing behavior of both cell types.

### Example III

### NRP2 is Upregulated During Melanoma-Endothelial Cell Communication and is Expressed in Metastatic Melanomas

NRPs are transmembrane glycoproteins that modulate the development of the nervous and vascular systems [17-19]. They function as co-receptors interacting with the vascular endothelial growth factor (VEGF) receptors and the plexins, and bind two known ligands with distinct functions: class 3 semaphorins, involved in axonal guidance; and VEGF family members known to promote angiogenesis. Blocking NRP2 function has recently been shown to inhibit tumor metastasis through effects on lymphendothelial cell migration and tumor-associated lymphangiogenesis [20]. NRPs have also been implicated in tumorigenesis as they are expressed in a variety of cancers [17, 21-27]. Additionally, NRP2 has recently been shown to regulate processes essential for melanoma metastasis and angiogenesis in vivo [30], but the mechanisms of these regulations are not clear. Therefore, the potential role of NRP2 in regulation of the phenotypic cell responses to co-culture conditions was evaluated using the various methods described above.

NRP2 protein expression was evaluated in melanoma cells during co-culture by immunoblotting using the sc-5542 antibody (Santa Cruz) under standard conditions. Samples were run on acrylamide gels, and transferred onto an Immobilon-P membrane. The membranes were then probed with commercially available NRP2 antibodies available from Santa Cruz™ (Santa Cruz, Calif.). The immunoblotting results confirm an increased expression of NRP2 in the co-cultured melanoma cells (FIG. 2B).

Examination of NRP2 expression in primary human melanoma tissues demonstrated specific, high-level expression of NRP2 within the tumor parenchyma in 5/5 metastatic melanomas evaluated (FIGS. 2C-E). Furthermore, such staining was consistent with tumor staining by the melanocyte marker, Melan-A (2F). Therefore, NRP2 is expressed in metastatic melanomas in vivo.

### Example IV

### NRP2 Mediates Melanoma Cell Proliferation

Since NRP2 can interact with VEGF and semaphorin signaling, which are known to regulate cell proliferation and/or migration, the functional significance of NRP2 over-expression in melanoma-endothelial co-cultures was investigated using an NRP2-neutralizing antibody [28, 29]. The antibody evaluated in the studies was generated against amino acids 560-858 of NRP2 and would therefore block binding of both semaphorin and VEGF ligands.

### XTT Assay

Cells were plated at 3,000 cells/well in a flat bottom 96-well plate. A rabbit polyclonal NRP2 antibody (sc-5542, Santa Cruz) and normal rabbit IgG (sc-2027, Santa Cruz), or a mouse monoclonal NRP2 antibody (sc-13117, Santa Cruz) and normal mouse IgG (sc-2025, Santa Cruz) were used at a final concentration of 10 pg/ml for functional studies. XTT reagents (Cell Proliferation Kit II, Roche Applied Science) were added to a subset of wells every 24 hours for 8 days and color change was monitored by spectrophotometer. Cell numbers were extrapolated from standard curves. Cells were plated in the presence of antibody and medium was changed and antibody refreshed on days 2, 4, and 6. BrdU incorporation was measured using the BrdU Labeling and Detection Kit I (Roche Applied Science), following the manufacturer's instructions.

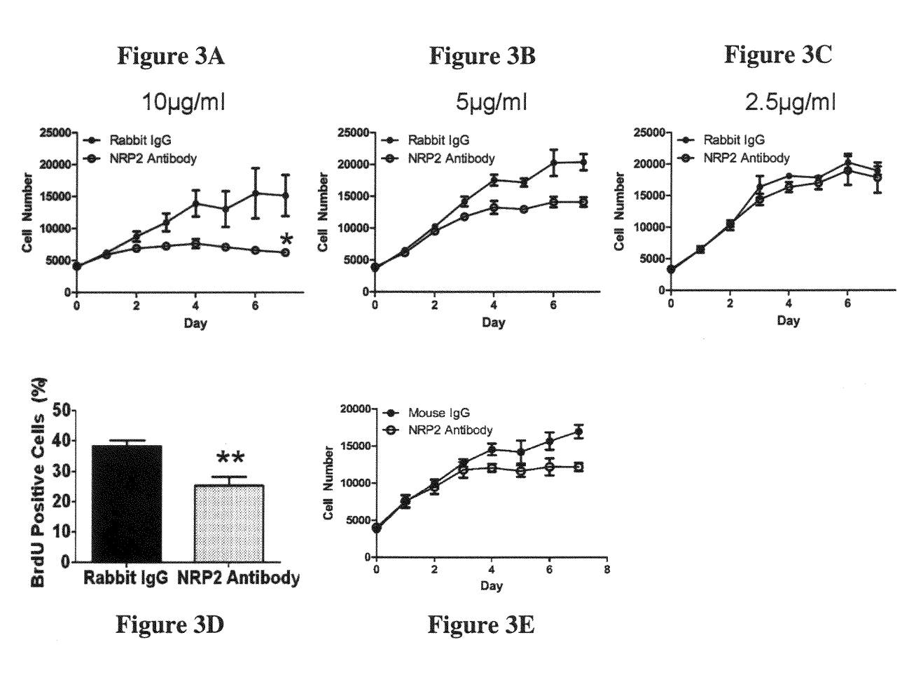

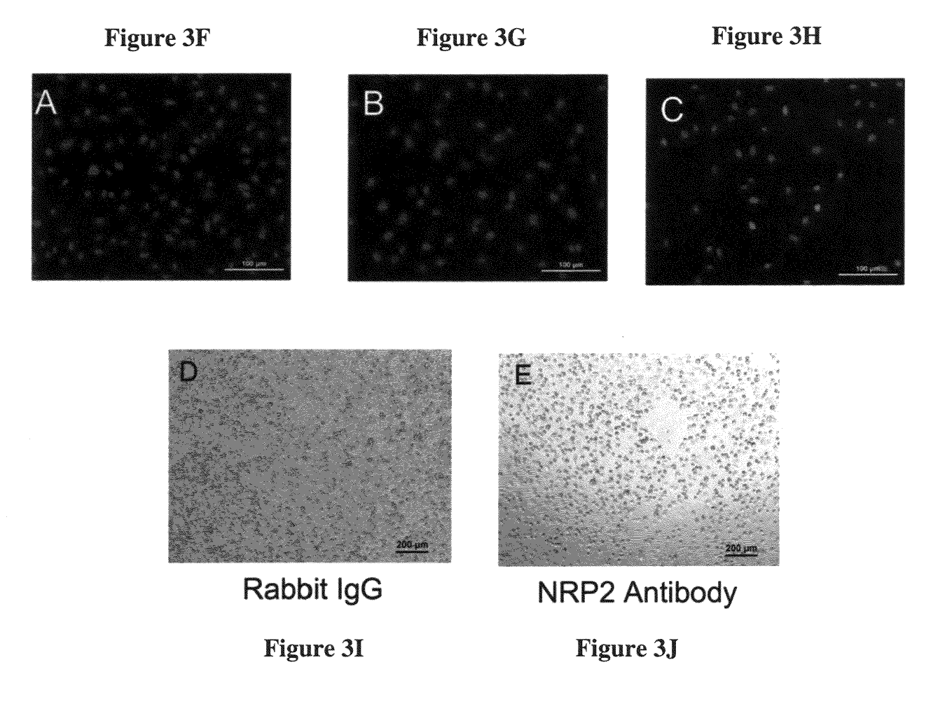

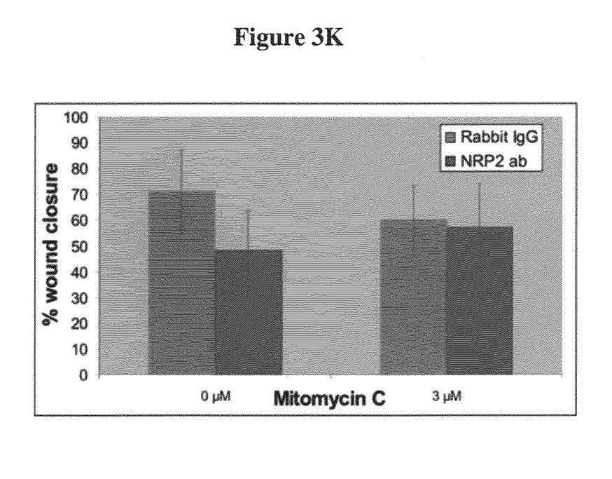

Interestingly, even in the absence of co-culture, NRP2-neutralizing antibody (H-300, sc-5542) severely decreased melanoma cell growth in vitro (FIG. 3A) suggesting that NRP2 is a critical mediator of melanoma cell proliferation. Studies with an alternative NRP2-neutralizing antibody (C-9, sc-13117) also confirmed a growth inhibitory role (FIG. 3E) and BrdU incorporation assays demonstrated significant growth inhibition at 48 hours following antibody treatment (FIG. 3D). Growth inhibition was titratable, as decreasing amounts of antibody had less of an inhibitory effect (FIGS. 3A-C).

### TUNEL Assay

Cells were plated at 3,000 cells/well in a flat bottom 96-well plate. A rabbit polyclonal NRP2 antibody (sc-5542, Santa Cruz) and normal rabbit IgG (sc-2027, Santa Cruz), or a mouse monoclonal NRP2 antibody (sc-13117, Santa Cruz) and normal mouse IgG (sc-2025, Santa Cruz) were used at a final concentration of 10 pg/ml for functional studies. For TUNEL assays, cells were plated in the presence of antibody, and TUNEL staining was performed following 48 hours of antibody treatment or 48 hours post-transfection. TUNEL staining was performed using the In situ Cell Death Detection Kit (TMR Red, Roche Applied Science).

Evaluation of cellular apoptosis by TUNEL staining demonstrated no notable increase in melanoma cell death (FIG. 3F-H). In addition, tumor cell morphology was not significantly altered following treatment with neutralizing antibody (FIGS. 3I and 3J).

### Scratch Assay

Cells were plated at 3,000 cells/well in a flat bottom 96-well plate. A rabbit polyclonal NRP2 antibody (sc-5542, Santa Cruz) and normal rabbit IgG (sc-2027, Santa Cruz), or a mouse monoclonal NRP2 antibody (sc-13117, Santa Cruz) and normal mouse IgG (sc-2025, Santa Cruz) were used at a final concentration of 10 pg/ml for functional studies. For the scratch assay, GFP-1205Lu cells were plated at 100% confluence in a 24 well plate. A 200 pl pipet tip was used to scratch a line in the cell monolayer, and the cells were washed 3 times in PBS. Mitomycin C was added at a final concentration of 0 pM or 3 pM in DMEM with 10% FBS. DAPI staining was used to visualize nuclei. All experiments were performed in triplicate. Micrographs for all the experiments were taken with a Nikon Eclipse microscope and analyzed using the MetaMorph software (Molecular Devices).

Antibody neutralization of NRP2 did not significantly alter cell migration in a scratch assay when proliferation was taken into account (FIG. 3K). These results suggest that NRP2 expressed by melanoma cells in homotypic cell culture specifically supports cell proliferation, possibly by facilitating autocrine signaling by VEGF [35].

The XXT, the TUNEL, and the scratch assay results support a potentially critical role for VEGF in regulating melanoma cell growth in pure culture or co-culture environments.

### Example V

### NRP2 Promotes Collective Movement of HUVECs in Melanoma Co-Culture

Highly temporally resolved time-lapse imaging of HUVEC cell movement was used to investigate whether NRP2 could also control directed collective endothelial cell migration accompanying cell re-organization into branched and looped networks observed with all co-culture methods. For this purpose, the most controlled method of cell co-culture, the micropatterning method, was particular convenient due to the high degree of reproducibility of the initial conditions, i.e., the shape of the endothelial cell colony and its size.

Tumor cells were incubated with RFP-HUVECs for 48 hours and photographed using epifluorescence microscopy. A threshold value for images of RFP-HUVECs was determined using Otsu's method (Matlab's ‘graythresh’ function). Pixels above the threshold value corresponded to HUVECs and pixels below the threshold value corresponded to background (i.e., areas covered by co-cultured cancer cells). The RFP-HUVEC image was morphologically closed using a circle with a diameter of 100 pixels, then the metric of network formation was computed as the fraction of pixels below the threshold value. This method determined the fraction of the co-culture area that consists of large regions, at least 100 pixels in diameter, which were not covered by RFP-HUVECs. In the absence of network formation, such as when HUVECs are randomly dispersed in the co-culture, it is expected that few or no large regions in coverage by HUVECs, and the network formation metric is expected to be near zero. In the presence of network formation, such as when HUVEC cells aggregate and form patterns, many large regions not covered by HUVECs will appear, and the network formation metric would be expected to be positive.

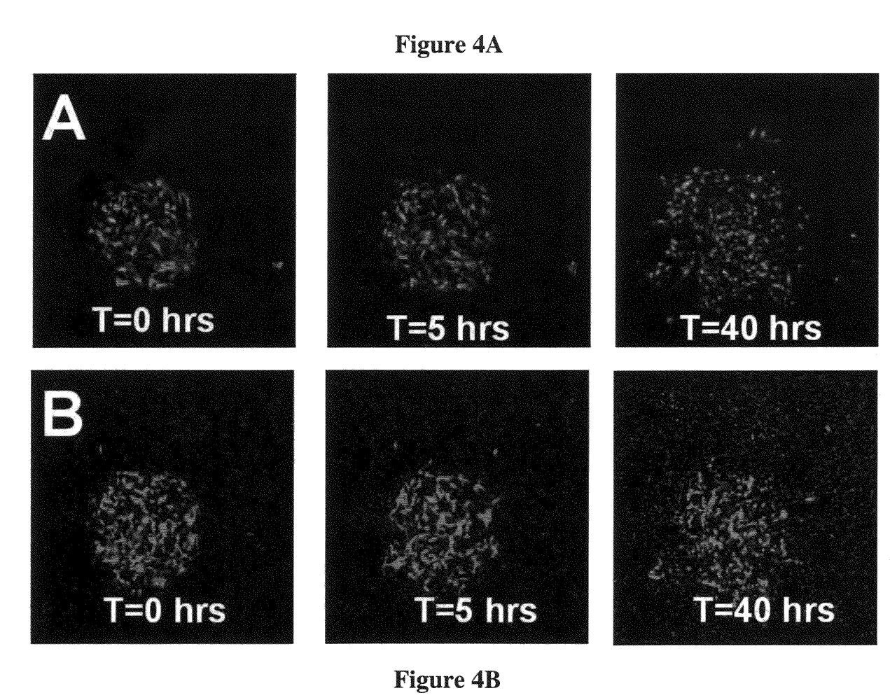

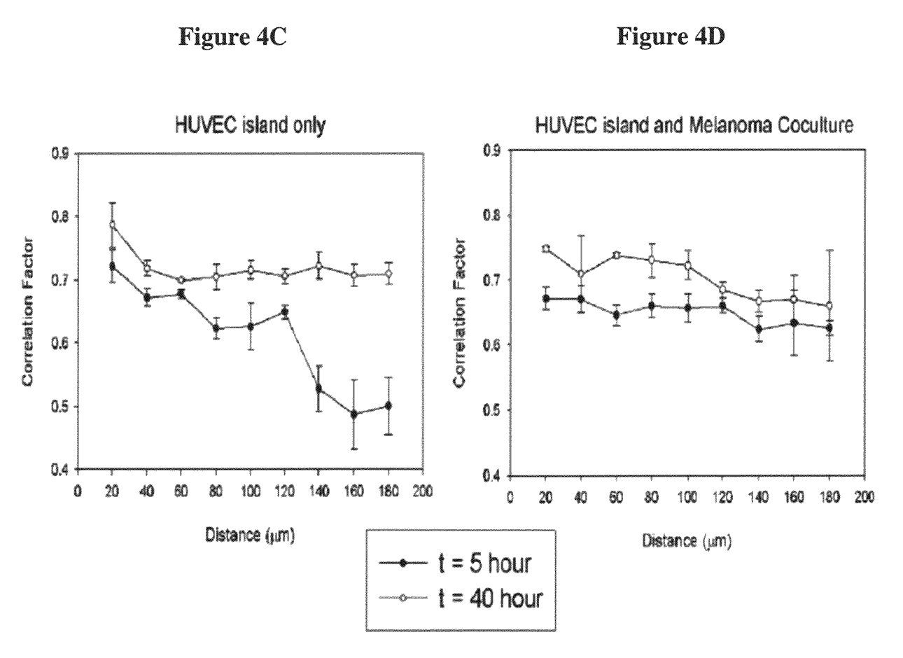

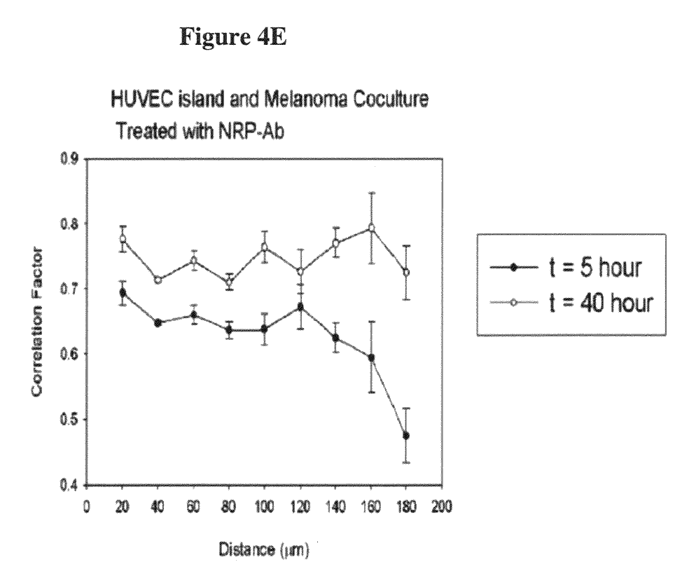

Collective movement of endothelial cells was quantified by estimating the correlation of cell velocities for various cell pairs C(r), as a function of the distance between the cells (r) (as described in [2]), for the control (HUVEC colony alone) and co-culture experiments at early (5 hours) and late (40 hours) time points (FIG. 4). Note that, although C(r) is expected to be 0 for sparse pure HUVEC colonies, it is commonly much higher for dense colonies, even in the absence of co-culture. This is in part due to correlated cell movement away from the center of the colony into available spaces for the distances measured (<200 pm).

Melanoma cells significantly enhanced directed HUVEC migration at the early but not at the late time point. Strikingly, NRP2-neutralizing antibody completely abolished this early enhancement effect by melanoma cells (FIG. 4B, E), suggesting that NRP2 plays a critical role in mediating endothelial cell organization during the initial stages of the cellular network formation. Furthermore, the effect of the NRP2-neutralizing antibody was most pronounced at relatively large cell-cell distances (>120 pm). These observations suggest that the NRP2 effects on endothelial cell migration during co-culture are likely to be long range, and may influence cell behavior through a diffusion-based process rather than contact-based cell-cell interactions, in agreement with the results of the experiments with conditioned media. These results further imply that the branching patterns observed in heterotypic co-cultures (FIGS. 1B-D) may, at least in part, be dependent on the initial (over the first 5 hrs.), NRP2-dependent correlation in cell movement direction and speed. NRP2, likely in its secreted form, might assist melanoma cells in recruitment of endothelial cells ensuring that this recruitment results in functional new vasculature, and thus both enhance melanoma survival and provide routes for metastasis. Given the in vivo [20] and in vitro evidence regarding NRP2 functions in melanoma growth and tumor metastasis, NRP2 is an important mediator of melanoma-endothelial cell communication and is a worthwhile therapeutic target in treating melanoma.

### Example VI

### NRP2 Receptors and Ligands are Expressed in Melanomas

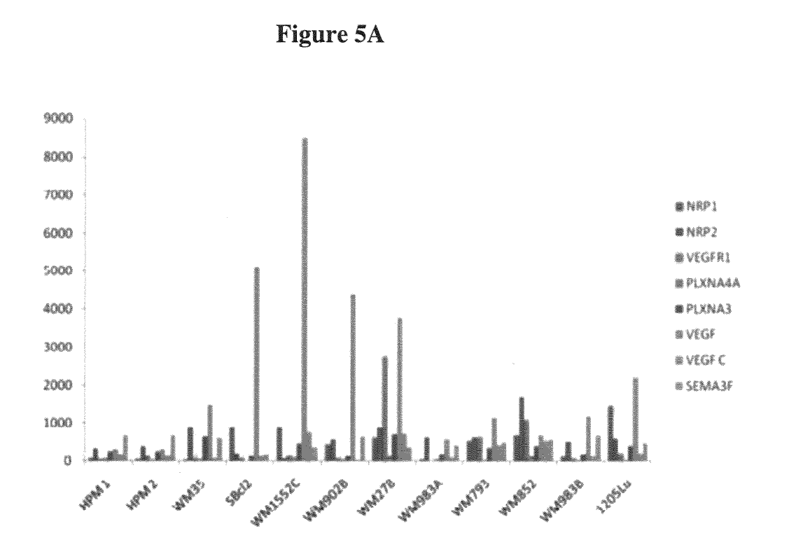

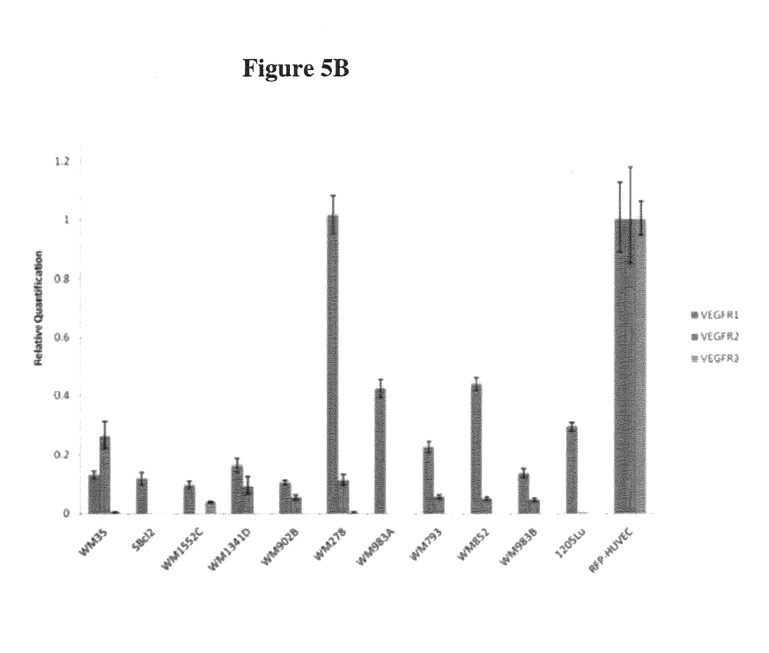

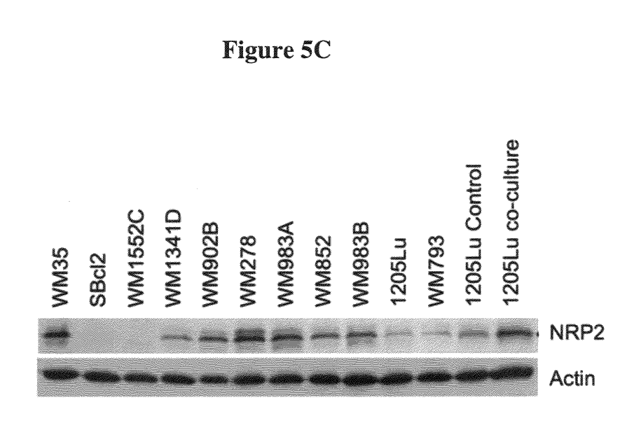

Expression of NRP2 ligands and co-receptors in a panel of melanoma cell lines was evaluated in order to define the pathways associated with the NRP2 function in melanomas. Previous gene expression studies of melanoma cell lines from varying stages of malignant progression provided the molecular signatures associated with melanoma progression [5]. These data were mined to investigate expression of NRP2, its homologue NRP1, and its binding partners: VEGFR1, plexinA4A, plexinA3, VEGF-A, VEGF-C, and Sema3F. NRP2 expression was detected in all stages of melanoma with lower expression noted in 2 of 3 radial growth phases (FIG. 5A). The expression of VEGF-A was elevated in early versus late stage melanomas (FIG. 5A), while low-level expression of Plexins, Sema3F, and VEGF-C was seen in all melanoma cell lines evaluated and Nrp1 expression was virtually absent (FIG. 5A).

The relative expression levels of VEGFR1, VEGFR2, and VEGFR3 in human melanoma cell lines and RFP-HUVECs were determined by quantitative real time polymerase chain reaction (qRT-PCR) analysis. The following primer oligonucleotides were used:

VEGFR1 forward
(5′-GCACCTTGGTIGTGGCTGAC-3′)
VEGFR1 reverse
(5′-GAGCAAGGATGAAGGCACTC-3′)
VEGFR2 forward
(5′-CATCACATCCACTGGTATTGG-3′)
VEGFR2 reverse
(5-GCCAAGCTTGTACCATGTGAG-3′)
VEGFR3 forward
(5′-CCCACGCAGACATCAAGACG-3)
VEGFR3 reverse
(5′-TG CAGAACTC CAC G AT CAC C-3′)
GAPDH forward
(5′-CATGAGAAGTATGACAACAGCCT-3)
GAPDH reverse
(5′-AGTCCTTCCACGATACCAAAGT-3′).

RNA was extracted using the RNeasy Mini Kit (Invitrogen). 3 pg of RNA for each sample was used for cDNA synthesis using SuperScript First-Strand Synthesis System according to manufacturer's instructions (Invitrogen). The qRT-PCR was carried out in a total volume of 20 pL per reaction, each containing 1 pL cDNA, 10 pL SYBR Green PCR master mix (Applied Biosystems), 1 pL of 10 pM forward primer, and 1 pL of 10 pM reverse primer. DNA was amplified using the following parameters: 50° C. for 2 minutes, 95° C. 10 minutes, followed by forty cycles of 95° C. for 25 seconds, 60° C. for 31 seconds, and 72° C. for 1 minute. VEGF receptor gene expression was normalized using reference primers against GAPDH. The raw quantification data for the melanoma cell lines was calibrated against RFP-HUVEC VEGF receptor gene expression.

All cell lines examined expressed high levels of VEGFR1, with variable low-level VEGFR2 and VEGFR3 expression (FIG. 5B). NRP2 protein expression was also at the highest level in vertical growth phase melanomas, with little detectable protein in 2 of 3 of early (radial) growth phase melanomas (FIG. 5C). These results suggested that NRP2 can exercise its effects through its natural binding partners, including, most notably, VEGF receptors.

### Example VII

### Patterning Interactions Between Tumor Cells and Endothelial Cells Varies with Tumor Type

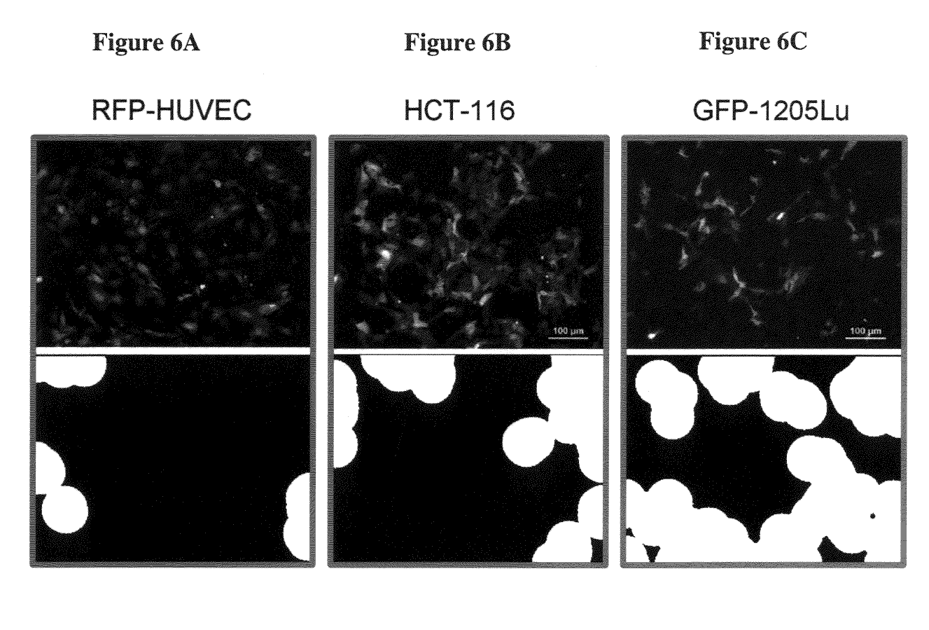

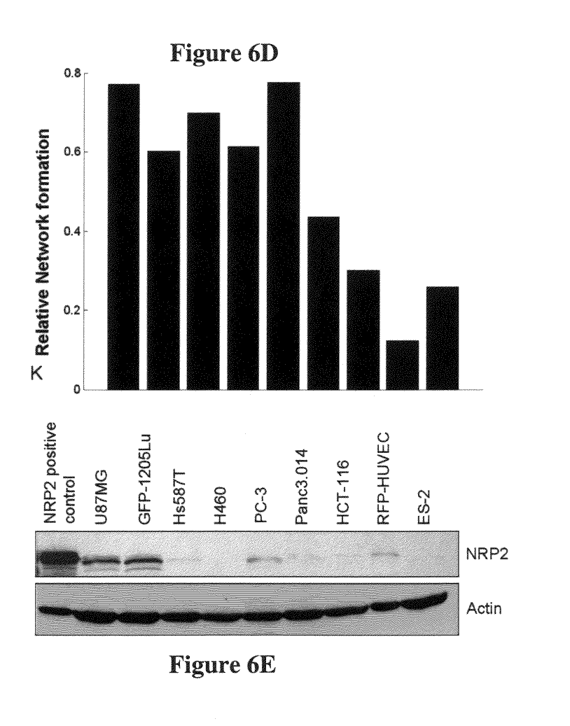

Using the gap interface co-culture method, the ability of various tumor cells to promote HUVEC patterning was evaluated (FIG. 6). Patterning was assessed by quantifying the circular areas formed by networks of HUVEC cells using an automated image-analysis system (FIG. 6A-C, bottom). Interestingly, a range of HUVEC pattern induction by various tumor cell lines was observed, with mild to moderate patterning induced by ovarian, colon, and pancreatic cancer cells, and the strongest patterning induced by non-small cell lung cancer, prostate cancer, breast cancer, glioblastoma, and melanoma cells (FIG. 6D, top). Although there was not a strict correlation between tumor cell expression of NRP2 and patterning (FIG. 6D), 3 of the top 5 patterning-associated tumor cell lines expressed significant levels of NRP2.

These studies suggest that several cancer cell types, including melanoma and glioblastoma cells, can have high levels of NRP2 expression, which in turn may affect communication with endothelial cells to promote endothelial cell patterning. It is striking that NRP2 functions at an interface of neural cell and endothelial cell fates, and that melanoma cells elicit such a strong response to communication with endothelial cells through this co-receptor. As the cell of origin for melanoma is the neural crest-derived melanocyte, the strong communication network for these tumor cells with their associated vasculature have features in common with the interactions between neural and endothelial cells. Indeed, the striking ability for melanomas to metastasize at early stages of development may relate to their genetic memory of developmental cues associated with neural crest migration. Neuropilins, cellular receptors that function at this neural-endothelial cell interface, are likely to be critical mediators of intercellular communication, which serves a critical developmental role in early life but may also recapitulate developmental cues to promote tumor angiogenesis and metastasis. Thus, NRP2 is a mediator of melanoma cell proliferation and melanoma-endothelial cell communication and is a critical therapeutic target in this disease.

### Example VIII

### NRP2 Expression in Cutaneous Melanomas and Benign Nevi

Tissues evaluated in immunohistochemical analyses were specimen microarrays established from formalin fixed, paraffin-embedded archival material derived from the archives of the Department of Pathology of Memorial Sloan-Kettering Cancer Center and collected under appropriate protocols. Tissues specimens used were not selected for outcomes measurements hence no annotations regarding patient clinical data are included. A mixture of pigmented and non-pigmented, spindle and epithelioid, as well as desmoplastic melanomas were included in the melanoma tissue microarray (TMA). These histologic parameters have previously been correlated with the expression of melanocyte differentiation antigens. Benign nevi were obtained from the Johns Hopkins Department of Pathology archives under an IRB-approved protocol. Immunohistochemistry was performed using the EnVision System HRP (DakoCytomation). The slides were deparaffinized and rehydrated using a graded alcohol series. Citrate buffer (pH 6.0, 10 mM) was used for antigen retrieval. Using the capillary gap method, the sections were incubated overnight with rabbit polyclonal antibodies against NRP2 (SC-5542, Santa Cruz Biotechnology). A dilution of 1:50 was found to provide the optimum staining results. 3-amino-9-ethyl carbazole (AEC) was used as a chromogen and the sections were counterstained with hematoxylin.

Most cases for evaluation had 3 tissue sections from the same specimen available, while others had only one or two. Each tissue section had two pathology interpretations for analysis. When more than one section was available for a case, and the pathology interpretations were not concordant, an average interpretation was taken to represent that case. The final results of each case for each tumor type were then averaged to determine the average percentage stained and intensity of each tumor type by pathologist review. By averaging the total sections for each case, no case with multiple sections carried more weight than another with only one section, and it was also a more accurate representation of NRP2 staining throughout a case.

The tissues used for analysis were normal tissues, various types of non-melanocytic tumors, and various cutaneous melanomas. The TMA slides were scanned and digitized using the Bacus Labs Inc. Slide Scanner (BLISS, Bacus laboratories, Lombard, Ill.). The images were uploaded into the TMAJ database for evaluation. Tissues that were not considered representative samples of the tissue being studied were removed from the analysis. The slides were examined qualitatively and tissue staining was estimated and graded as follows: less than 20%, 20-60%, or greater than 60% of the tissue present. The intensity of NRP2 staining was also scored from 0 to 3 with 0 having no NRP2 staining and 3 having the highest intensity. The extent and intensity of staining was documented and compared to control samples that were strongly positive for NRP2.

FRIDA (FRamework for Image Dataset Analysis), a custom open source image analysis software package, was used for the analysis of RGB color image datasets, and for image analysis, including those generated from scanning of tissue microarray slides. Hue saturation and brightness (HSB) segmentation ranges for red staining and hematoxylin alone (nuclei not staining red) were defined from the tissue microarray image set. Using the specific color pixel definitions for “total tissue,” “positive NRP2 staining tissue,” “stained nuclei,” and “remaining tissue,” the Java software program analyzed images with the selected color pixels to quantify positive staining. In the study, the entire tissue area was defined as “tissue area,” stained nuclei were defined labeled “nuclei,” and the specific NRP2 staining color positive mask was defined as “NRP2 area.” Since nuclei were not expected to be stained according to the preliminary testing studies, and nuclei can be very large in tumor cells, the remaining tissue area that was expected to stain for NRP2 was redefined as tissue that is in the “tissue area” but not in the “nuclei” and subsequently labeled “cytoplasm.” By redefining the total tissue area without nuclei, a more accurate calculation based on total possible staining area for NRP2 was established. The percentage of staining was calculated by the FRIDA program as the “NRP2 area”/“cytoplasm” (total tissue area without nuclei). The results of the FRIDA computer analysis along with the pathologist evaluations were analyzed using the R version 2.6 statistical software program. A Welch two sample t-test with unequal variances was used to statistically evaluate the FRIDA NRP2 staining differences between melanocytic and non-melanocytic tumors.

### Analysis of Normal Tissue

TABLE 3
Tissue microarray immunohistochemical
analysis of normal tissue with NRP2

| | Average NRP-2 | | Cases Positive/ |
|---|---|---|---|
| Normal Tissues | percent staining | Intensity | Cases Examined |
| Esophagus | − | | 0/2 |
| Stomach | − | | 0/1 |
| Small Bowel | − | | 0/2 |
| Appendix | − | | 0/4 |
| Colon | − | | 0/2 |
| Gallbladder | − | | 0/1 |
| Lung | − | | 0/7 |
| Parotid | − | | 0/2 |
| Omentum | − | | 0/2 |
| Thymus | − | | 0/2 |
| Adrenal | − | | 0/4 |
| Lymph node | − | | 0/1 |
| Bladder | − | | 0/3 |
| Vaginal tissue | − | | 0/1 |
| Thyroid | − | | 0/3 |
| Amnion | − | | 0/2 |
| Tonsil | − | | 0/2 |
| Endometrial | + | low | 3/3 |
| Pancreas | + | low | 2/2 |
| Prostate | + | moderate | 2/2 |
| Spleen | + | moderate | 3/4 |
| Breast | + | moderate | 2/3 |
| Muscle | ++ | moderate | 3/3 |
| Fallopian tube | ++ | moderate | 2/2 |
| Liver | ++ | moderate | 2/2 |
| Skin | ++ | high | 2/3 |
| Placenta | ++ | high | 2/2 |
| Kidney | ++ | high | 3/3 |
| Testes | +++ | high | 3/3 |

− Negative; +, <20% of tissue positive; ++, 20 to 60% of tissue positive; +++, >60% of tissue positive by pathologist review.

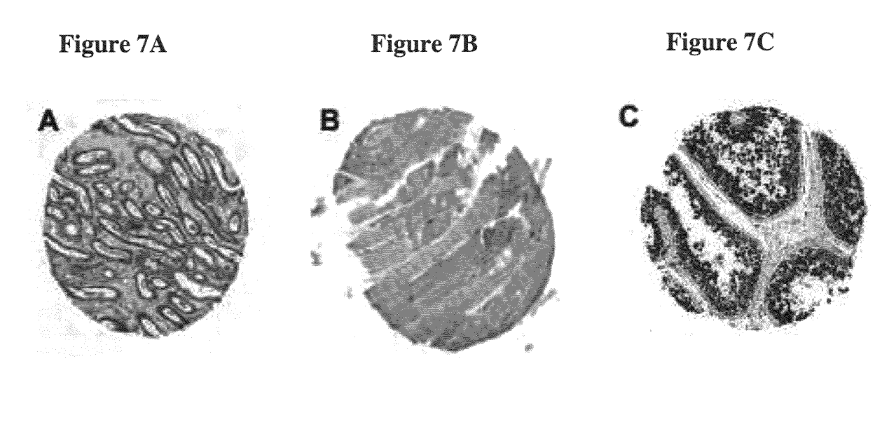

The qualitative immunohistochemical analysis of NRP2 staining for normal tissues is found in Table 3. NRP2 staining was notable in liver, kidney, fallopian tubes, pancreas, placental tissue, testis, prostate, striated muscle cells, specimen specific breast ductal tissue, skin epidermis, spleen, and endometrial tissue (FIG. 7). All samples of normal liver were mildly NRP-2 positive with scattered hepatocyte staining. The majority of normal kidney tissue samples showed strong NRP2 staining of the glomerular endothelial cells, collecting tubules and collecting ducts. The mucosal lining cells of fallopian tubes stained intermittently positive in all specimens. Placental specimens showed intense, intermittent NRP2 staining of the syncytiotrophoblast cells of the placental villi. These same specimens also showed intermittent staining of the fetal capillaries within the villous cores. Breast tissue showed selective NRP2 breast duct epithelial cell staining, based on the core sample. Striated muscle cells showed moderate scattered NRP-2 staining in all available specimens. The skin specimens stained strongly positive for NRP2 only within the epidermal layer and some specimens possessed minimal staining of the basal cell layer of the epidermis. Endometrial tissue stroma cells and glandular cells stained intermittently positive for NRP2 within their nuclei with minimal staining of the cytoplasm. The testis stained strongly positive for NRP2 within the epithelium of the seminiferous tubules. Prostate specimens stained mildly positive for NRP2 in the prostatic glandular epithelial cells, predominantly as a light staining hue to the foamy cytoplasm. All other tissue types were negative for NRP2.

### Analysis of Tumors and Melanomas

TABLE 4
Tissue microarray immunohistochemical analysis for
NRP2 staining of various non-melanocytic tumors

| | Average NRP2+ | | Computer |
|---|---|---|---|
| | By Pathologist | Cases Positive/ | Mean + |
| Tumor Type | Review | Cases Examined | (%) |
| Breast carcinoma, | + | 2/5 | 2.9 |
| lobular | | | |
| Breast carcinoma, | + | 3/5 | 5.1 |
| ductal | | | |
| Leiomyosarcoma | + | 2/3 | 9.9 |
| Ovarian mucinous | − | 0/1 | − |
| Ovarian serous | − | 0/4 | − |
| Colon adenocarcinoma | + | 2/4 | 3.7 |
| Transitional cell | + | 2/3 | 9.7 |
| carcinoma | | | |
| Lung adenocarcinoma | − | 0/1 | − |
| Liposarcoma | − | 0/4 | − |
| Spindle cell sarcoma | − | 0/1 | − |
| Malignant fibrous | − | 0/4 | − |
| histiocytoma | | | |
| Non-small cell lung | − | 0/1 | − |
| ca. (squamous) | | | |
| Renal cell carcinoma | +++ | 4/5 | 49.9 |
| (clear cell) | | | |

− Negative; +, <20% of tissue positive; ++, 20 to 60% of tissue positive; +++, >60% of tissue positive by pathologist review.

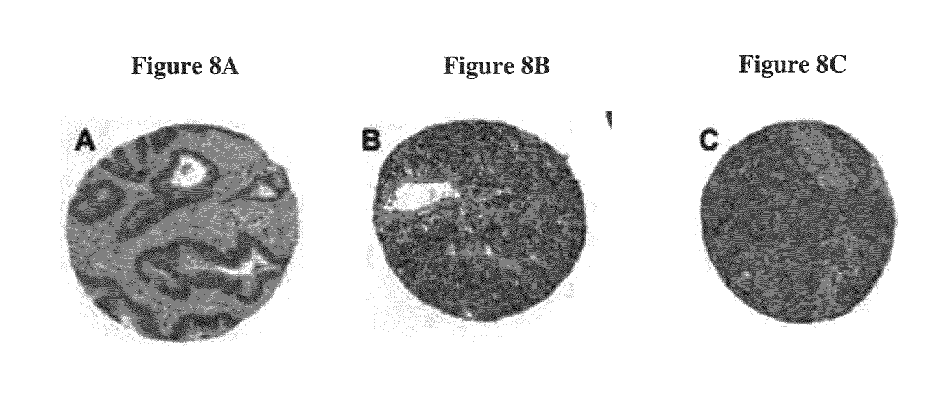

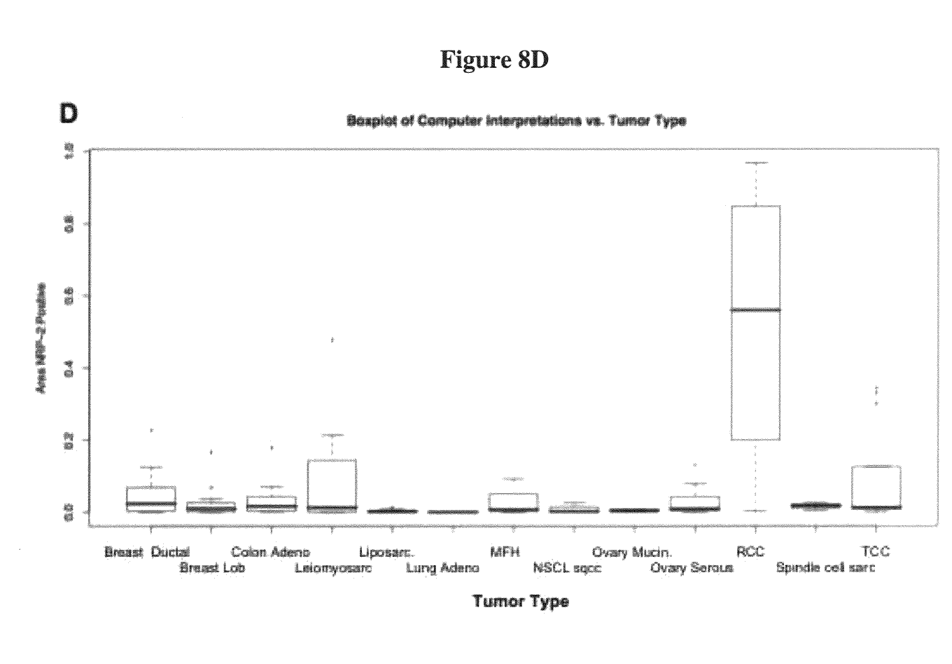

Immunohistochemical staining for NRP2 was evaluated for a variety of tumors (Table 4, FIG. 8). Tumors of the breast stained specimen-specific for NRP2 with 2/5 lobular breast carcinoma cell cases staining mildly positive, and 3/5 ductal breast carcinoma cases staining NRP2 positive, but not across all sections. Leiomyosarcoma specimens also stained in a case-specific manner for NRP2 with one specimen staining negatively, and two others staining positively. Four of the five renal cell carcinoma (clear cell) cases stained positively for NRP2, and most sections stained strongly positive. Colon adenocarcinomas stained case specifically positive for NRP2, with 2/4 cases staining mildly positive for NRP2. The colon adenocarcinomas that stained positively had scattered intranuclear and crypt cell cytoplasmic staining. Transitional cell carcinoma of the bladder staining for NRP2 was also case-specific, with 2/3 cases staining mildly positive. All ovarian mucinous, ovarian serous, lung adenocarcinoma, liposarcomas, spindle cell sarcomas, non-small cell lung cancer (squamous cell carcinoma), and malignant fibrous histiocytoma cases were negative for NRP2.

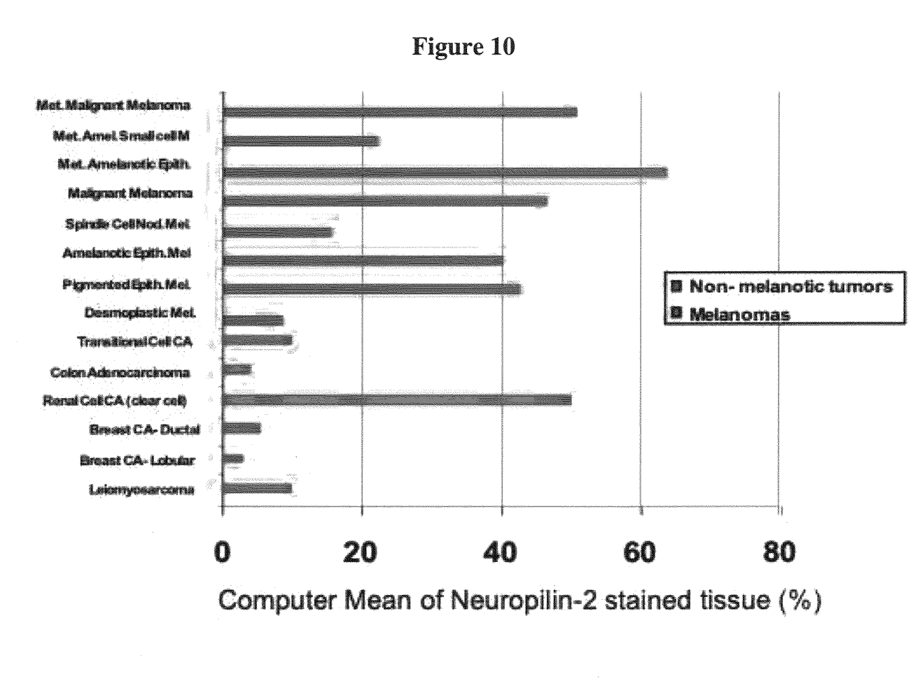

The FRIDA computer analysis of the variety of tumors (FIG. 8D) indicated the mean percentage of all stained tumor tissues was 10.4%. Renal cell carcinoma had the highest mean percent stained with 49.9%. This result is not surprising as normal renal tissue stains strongly positive for NRP2 in renal glomeruli and tubules. The computer analysis of the remaining positive NRP2 tumors calculated the average percentage stained as follows: breast carcinoma ductal 5.1%, breast carcinoma lobular 2.9%, colon adenocarcinoma 3.7%, leiomyosarcoma 9.9%, transitional cell carcinoma 9.7% (Table 4, FIG. 8D, FIG. 10). These results are significantly lower than the majority of melanomas evaluated, as discussed below.

TABLE 5
Tissue microarray immunohistochemical analysis of malignant
melanomas and metastatic melanomas with NRP2

| | Average NRP2+ | | | Computer |
|---|---|---|---|---|
| | By Pathologist | Average | Cases Positive/ | Mean + |
| Tumor Type | Review | Intensity | Cases examined | (%) |
| Pigmented Epithelioid Melanoma | +++ | high | 8/8 | 42.6 |
| Amelanotic Epithelioid Melanoma | +++ | high | 6/6 | 40.2 |
| Spindle Cell Nodular Melanoma | +++ | moderate | 3/3 | 13.9 |
| Desmoplastic Malignant Melanoma | + | low | 5/5 | 8.5 |
| Malignant Melanoma | +++ | high | 17/18 | 46.4 |
| Met. Malignant Melanoma | +++ | moderate | 5/5 | 50.6 |
| Met. Amel Spindle Cell Malig. Mel. | ++ | low | 8/9 | 22.2 |
| Met. Amel Epithelioid Malig. Mel. | +++ | high | 8/8 | 63.5 |

− Negative; +, <20% of tissue positive; ++, 20 to 60% of tissue positive; +++, >60% of tissue positive by pathologist review.

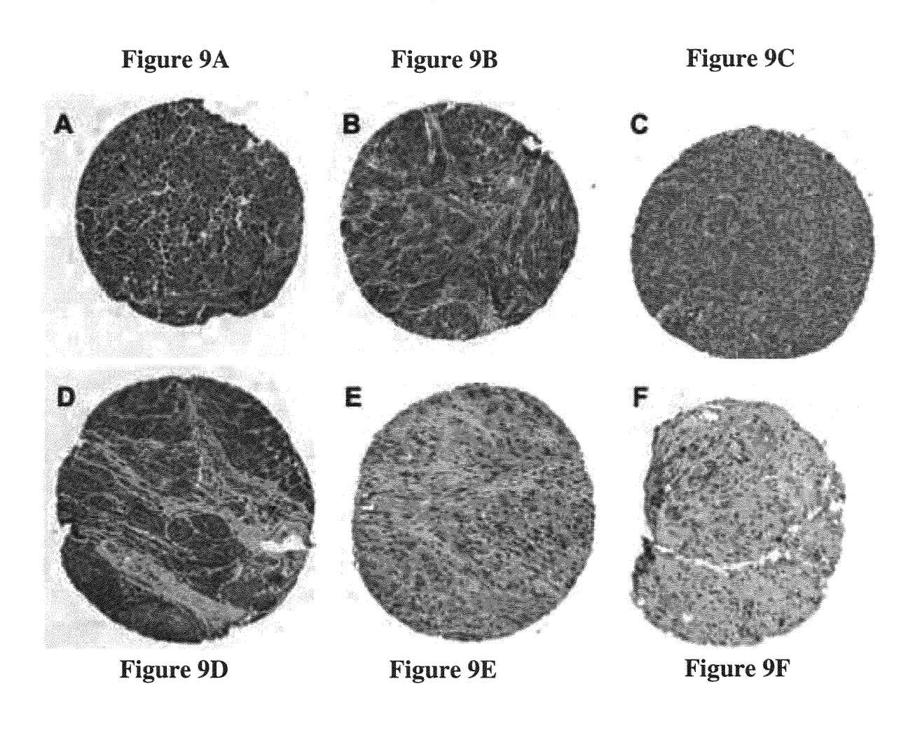

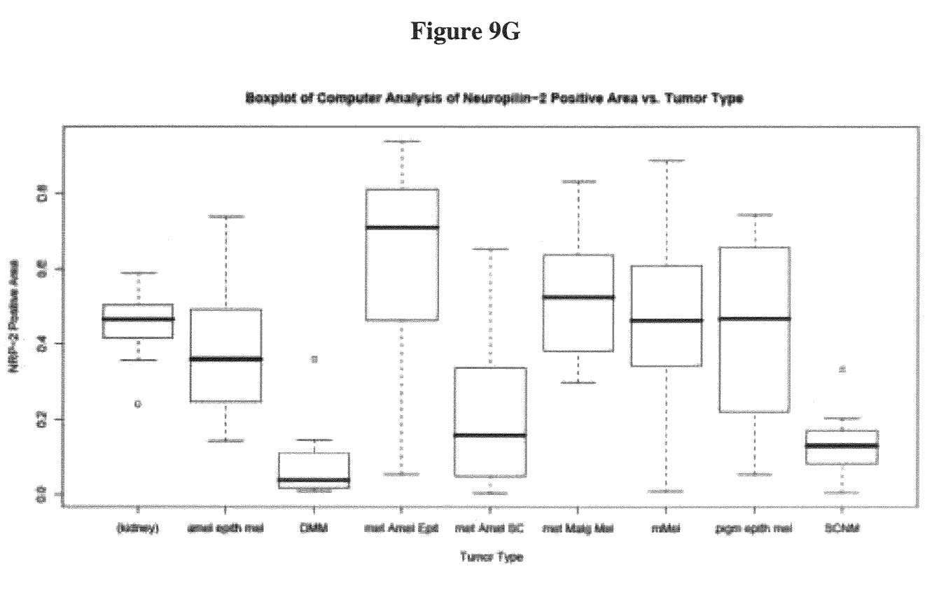

The immunohistochemical staining for NRP2 in various primary malignant melanomas and metastatic melanomas is shown in Table 5. Pigmented epithelioid melanomas demonstrated the most positive NRP2 staining with all cases staining positive (8/8) for NRP2 and most specimens staining greater than 60% by pathologist review with moderate to high intensity (Table 5, FIG. 9). Amelanotic epithelioid melanoma cases all stained positive for NRP2 (6/6) with the majority staining greater than 60% by pathologist review and all staining with moderate to high intensity. All of the spindle cell nodular melanoma cases also stained positive for NRP2 (3/3), with most showing moderate intensity and all having greater than 20% staining by pathologist review. Of all the melanoma cases, desmoplastic malignant melanoma had the mildest staining. All of the desmoplastic malignant melanoma cases were positive (5/5), and all stained less than 20% by pathologist review. The other malignant melanoma cases stained NRP2 positive in 17/18 specimens. A large majority of these stained greater than 20% of the field and staining intensity varied from mild to intense (FIG. 9).

The FRIDA analysis for the variety of melanomas stained for NRP2 showed a mean for all the tissues analyzed of 46.9% (FIG. 9G), a marked increase from the other tumors analyzed (FIG. 10). Desmoplastic malignant melanoma had the least percentage stained with an average of 8.5%. The computer analysis of the spindle cell nodular melanomas indicated a mean of 13.9% positive for NRP2. The epithelioid type melanomas had the greatest staining for NRP-2 with pigmented epithelioid melanoma expression having an average of 42.6% and amelanotic epithelioid melanoma having a mean percentage NRP2 positivity of 40.2%. Other melanomas had a mean NRP2 expression of 46.4% by computer analysis.

Metastatic melanomas were also analyzed for NRP2. The metastatic cases of malignant melanoma stained NRP2 positive in all five cases analyzed. All cases stained greater than 60% by pathologist review and all with moderate to intense staining. Metastatic amelanotic spindle cell melanomas stained NRP2 positive in 8/9 specimens and staining intensity varied from mild to intense. For cases of metastatic amelanotic epithelioid malignant melanoma, all cases stained positive (8/8), with all sections staining greater than 20% by pathologist review and the majority of staining greater than 60%. The majority of these cases stained intensely for NRP2.

The FRIDA analysis of metastatic melanomas was similar to that for non-metastatic melanomas. Metastatic amelanotic spindle cell melanomas had an average percent NRP2 staining of 22.2% by computer analysis; whereas metastatic amelanotic epithelioid cell melanoma and other metastatic malignant melanomas had higher percentages of 63.5% and 50.6% respectively (FIG. 10).

A Welch two sample t-test with unequal variances comparing the FRIDA results for melanocytic and non-melanocytic NRP2 expression was performed using the R statistical software package. The melanocytic tumors had a mean percent NRP2 staining of 40% versus the non-melanocytic tumor mean of only 10%. The difference in the means was 30%, and the 95% confidence interval for the difference in percent stained was (23.6, 35.5). The difference in the means was found to be statistically significant (p<0.0001).

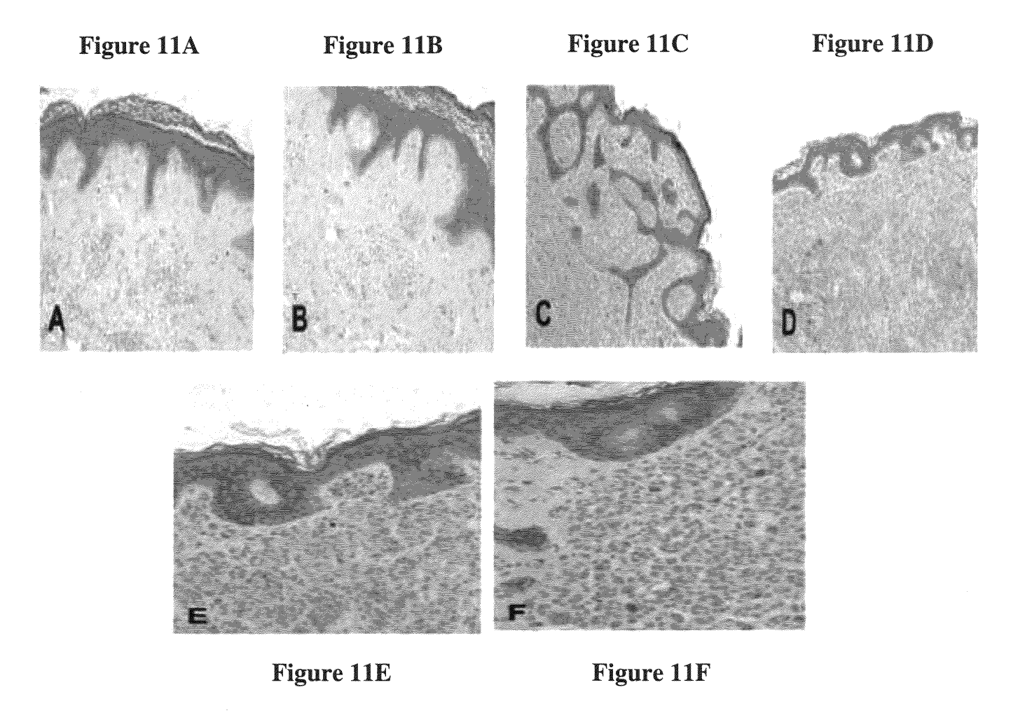

Staining of benign nevi for NRP2 was also evaluated in a limited number of tissue specimens. Notably, all benign nevi evaluated were negative for NRP2 staining while the suprabasal keratinocytes stained positively for NRP2 (FIG. 11). Interestingly, normal human melanocytes were also negative for NRP2 staining within the epidermis.

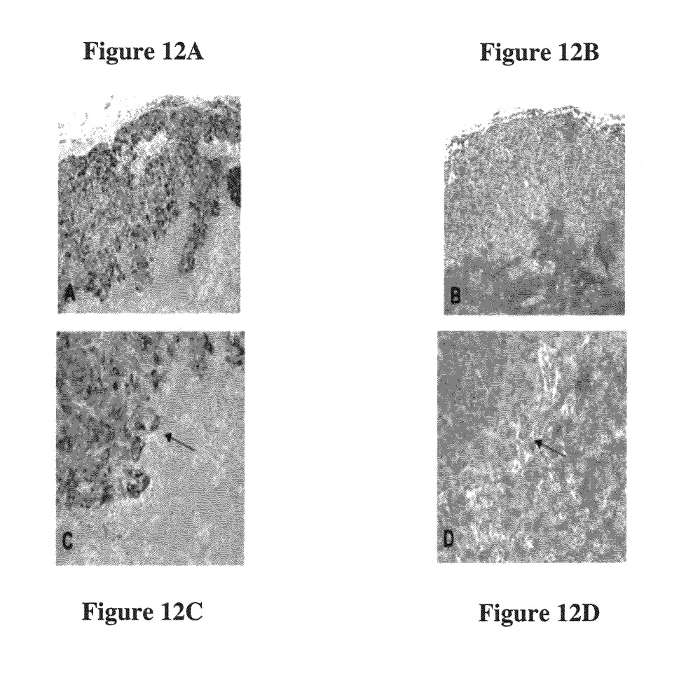

In order to confirm that expression of NRP2 was limited to melanoma cells seen in the metastatic setting, tumor specimens were evaluated for NRP2 and Melan-A. Expression of NRP2 matched the expression of Melan-A in metastatic melanomas suggesting specific expression of NRP2 in these cells (FIG. 12).

With 17/18 other malignant melanomas staining NRP2 positive on average greater than 60% by pathologist review, (46.4% by computer analysis), and with high intensity, while benign nevi are completely negative for NRP2 expression our study suggests that NRP2 may be a useful marker for melanoma and aid in the differentiation of benign versus malignant melanocytic tumors. In addition, given the differential expression of NRP2 in benign and malignant melanocytic tumors, NRP2 may be a useful prognostic biomarker in melanoma. Given that NRP2 can be expressed in a secreted form, detection of this secreted protein may also be useful as a surrogate melanoma marker for the identification of patients with occult metastatic disease.

### Example IX

### NRP2 is Expressed as a Secreted Protein

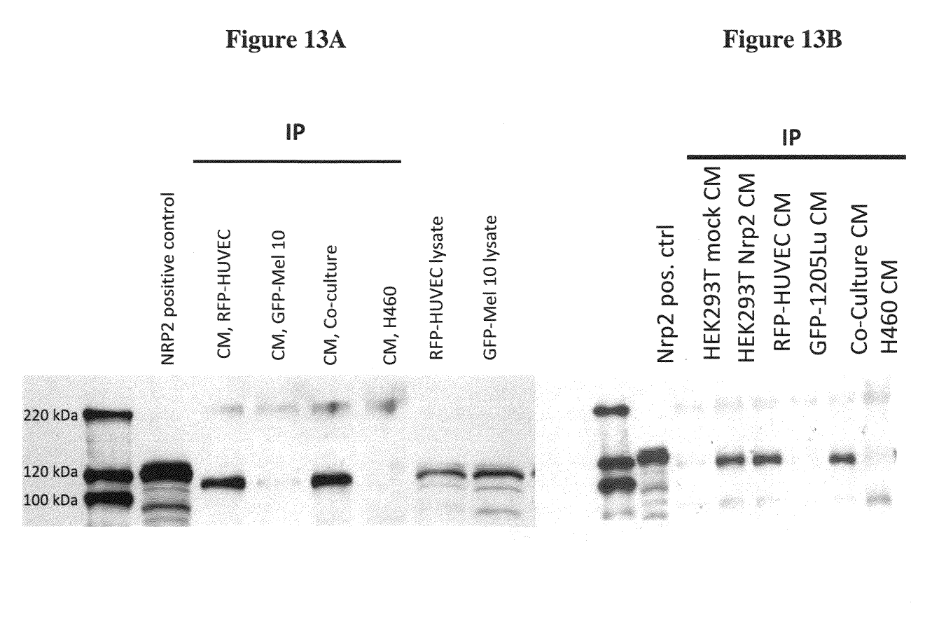

Since HUVEC patterning was dependent on tumor-associated secreted soluble factors, and since NRP2 may exist in both a secreted form and as a cell surface receptor [19], the expression of NRP2 in conditioned media derived from either HUVECs alone, GFP-1205Lu cells alone, or from cells co-cultured using the random mixture method were evaluated (FIG. 13A). A significant increase in the concentrations of NRP2 in the media conditioned both by RFP-HUVECs and by the random mixture method co-culture system versus the medium conditioned by HUVECs alone was observed. These results suggested that NRP2 could be produced by melanoma cells in at least partially soluble form, thus being a potentially interesting putative mediator of paracrine melanoma-endothelial cell-cell communication.

These results were confirmed and further supported by the IP-western analysis of NRP2 expression from conditioned media collected from mock and NRP2 transfected HEK293T cells, RFP-HUVECs, GFP-1205Lu melanoma cells, HUVEC-1205Lu co-cultures, and H460 (NRP1+/NRP2−) lunc cancer cells (FIG. 13B).

### Example X

### Detection of Recombinant Human NRP2 by ELISA

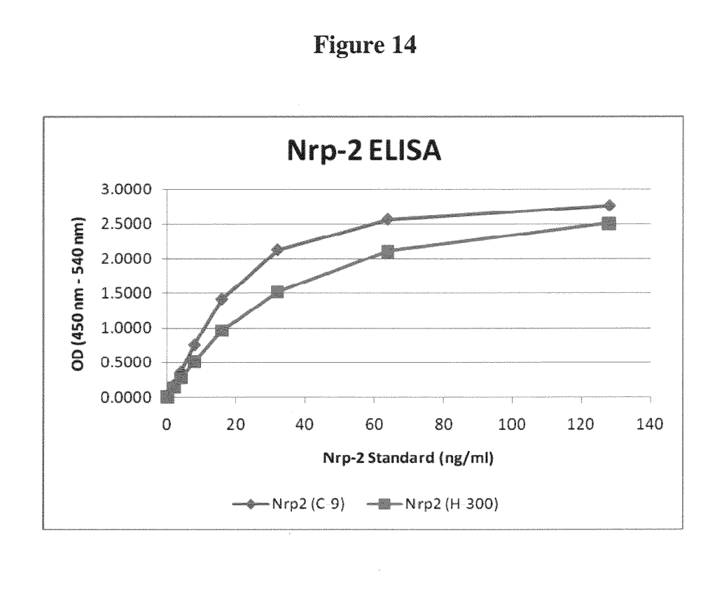

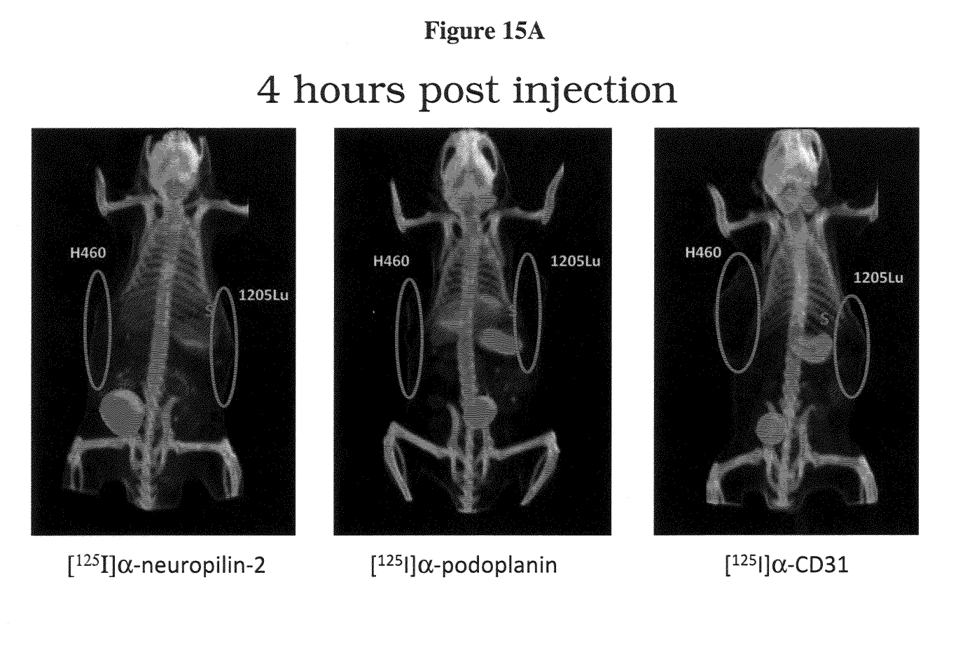

Varying concentrations of recombinant human NRP2 (R&D Systems, 2215-N2) were plated into a 96-well plate. A rabbit polyclonal NRP2 antibody (Santa Cruz™, sc-5542) and a mouse monoclonal NRP2 antibody (Santa Cruz™, sc-13117) were able to detect the varying concentration of recombinant human NRP2 (FIG. 14). Therefore, ELISA can be used to detect NRP2 levels in patient bloodstream, providing a sensitive tool for screening melanoma patients.

### Example XI

### In Vivo Melanoma Imaging

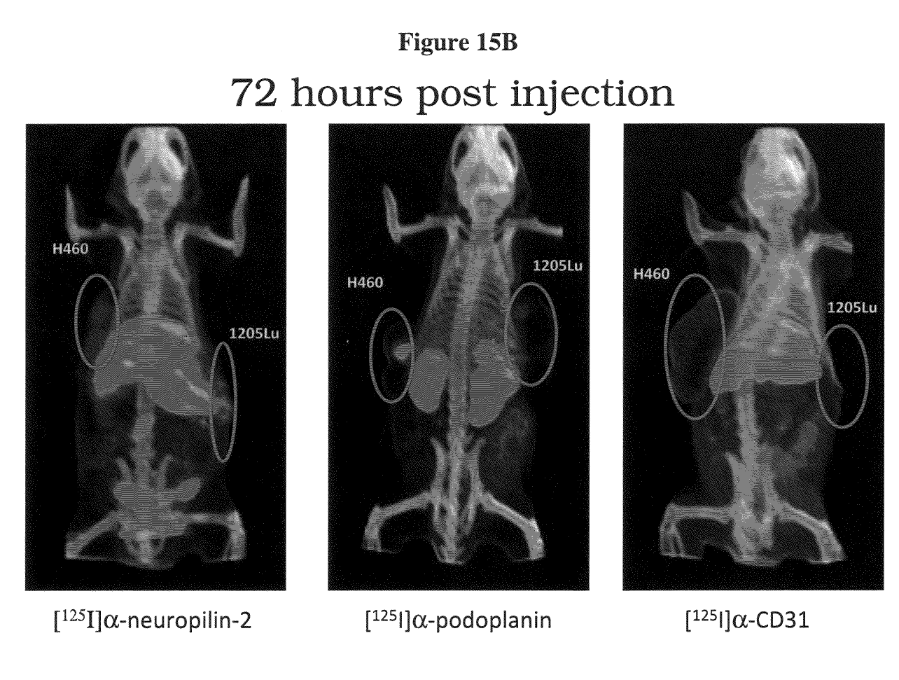

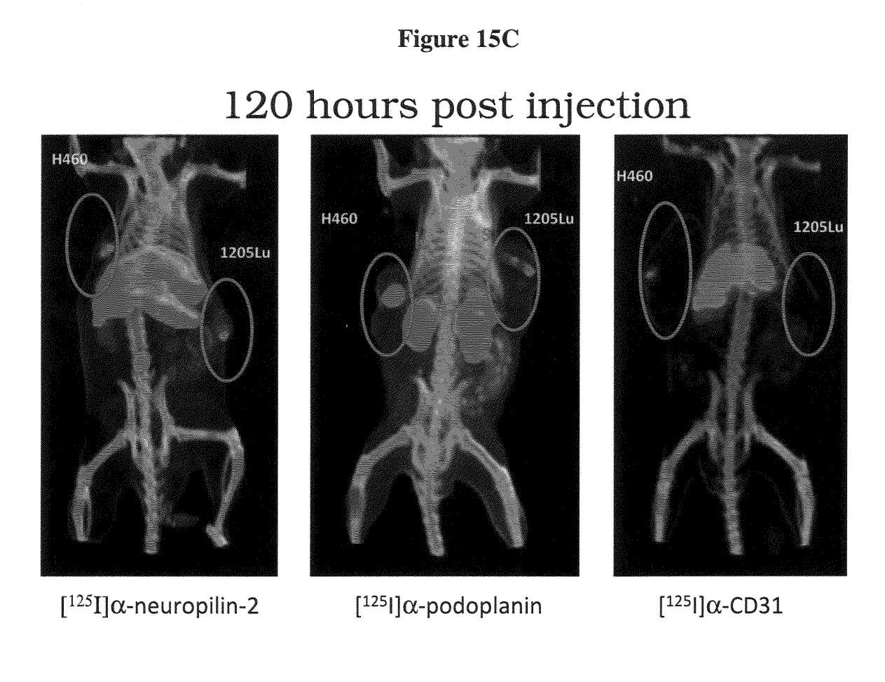

SCID mice were subcutaneously injected with 1205Lu melanoma cells and H460 lung cancer cells. Antibodies against NRP2 (Santa Cruz, sc-5542), podoplanin (to look at lymphatic vasculature), and CD31 (to look at blood vessel density) were radiolabeled with ¹²⁵I. Antibodies were intravenously injected and imaged after 4 hours (FIG. 15A), 72 hours (FIG. 15B), and 120 hours (FIG. 15C) post injection.

### Example XII

### Detection of Melanoma Cells Using FACS

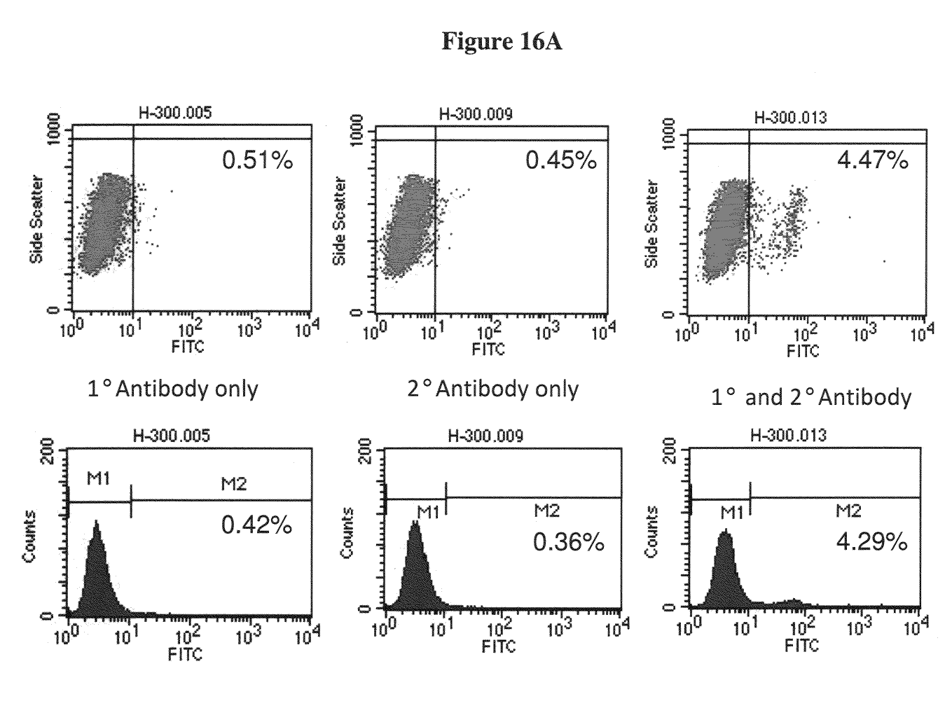

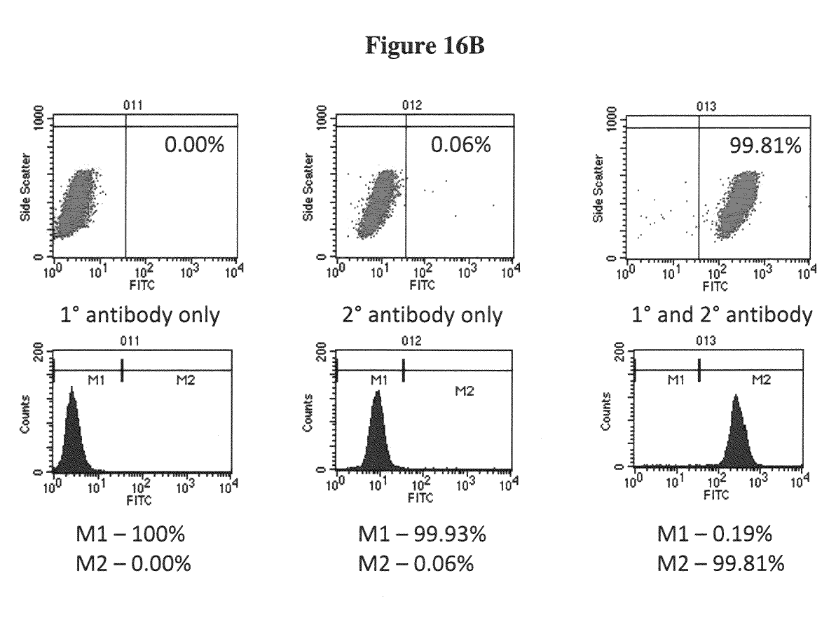

Experiments were performed to determine whether NRP2 expression could be detected on melanoma cells through NRP2 antibody labeling (Santa Cruz, sc-5542) and FACs analysis. Extracellular staining and FACS analysis of a vertical growth phase melanoma cell line reveals a subpopulation of cells that can be labeled (FIG. 16A). Intracellular staining for NRP2 demonstrates that over 99.8% of melanoma cells can be labeled and identified using an NRP2-specific antibody targeting the extracellular MAM domain of NRP2 (FIG. 16B). These results indicate that FACS analysis for NRP2 expression provides sufficient sensitivity and specificity to detect melanoma cells in a cell sample.

All publications, patents, patent applications, internet sites, and accession numbers/database sequences (including both polynucleotide and polypeptide sequences) cited herein are hereby incorporated by reference in their entirety for all purposes to the same extent as if each individual publication, patent, patent application, internet site, or accession number/database sequence were specifically and individually indicated to be so incorporated by reference.

References cited herein are listed below for convenience:
1. Ostuni E, Kane R, Chen C S, Ingber D E, Whitesides G M (2000) Patterning Mammalian Cells Using Elastomeric Membranes. 16(20): 7811.
2. Haga H, Irahara C, Kobayashi R. Nakagaki T, Kawabata K (2005) Collective movement of epithelial cells on a collagen gel substrate. Biophys J 88(3): 2250-2256.
3. Asaishi K, Endrich B, Gotz A. Messmer K (1981) Quantitative analysis of microvascular structure and function in the amelanotic melanoma A-MeI-3. Cancer Res 41(5): 1898-1904.
4. Endrich B, Hammersen F, Gotz A, Messmer K (1982) Microcirculatory blood flow, capillary morphology and local oxygen pressure of the hamster amelanotic melanoma A-MeI-3. J Natl Cancer Inst 68(3): 475-485.
5. Ryu B, Kim D S, Deluca A M, Alani R M (2007) Comprehensive expression profiling of tumor cell lines identifies molecular signatures of melanoma progression. PLoS One 2(7): e594.
6. Hendrix M J, Sensor E A, Hess A R, Seftor R E (2003) Vasculogenic mimicry and tumour-cell plasticity: lessons from melanoma Nat Rev Cancer 3(6): 411-421.
7. Velazquez O C, Herlyn M (2003) The vascular phenotype of melanoma metastasis. Clin Exp Metastasis 20(3): 229-235.
8. Dome B, Hendrix M J, Paku S, Tovari J, Timar J (2007) Alternative vascularization mechanisms in cancer: Pathology and therapeutic implications. Am J Pathol 170(1): 1-15.
9. Hess A R, Margaryan N V, Seftor E A, Hendrix M J (2007) Deciphering the signaling events that promote melanoma tumor cell vasculogenic mimicry and their link to embryonic vasculogenesis. role of the Eph receptors. Dev Dyn 236(12): 3283-3296.
10. Clark E A, Golub T R, Lander E S, Hynes R O (2000) Genomic analysis of metastasis reveals an essential role for RhoC. Nature 406(6795): 532-535.
11. Ridley A (2000) Molecular switches in metastasis. Nature 406(6795): 466-467.
12. Cha H J, Jeong M J, Kleinman H K (2003) Role of thymosin beta4 in tumor metastasis and angiogenesis. J Natl Cancer Inst 95(22): 1674-1680.
13. Adam F, Zheng S, Joshi N, Kelton D S, Sandhu A et al. (2005) Analyses of cellular multimerin 1 receptors: in vitro evidence of binding mediated by alpha1Ibbeta3 and alphavbeta3. Thromb Haemost 94(5): 1004-1011.
14. Yin Z, Noren D, Wang C J, Hang R, Levchenko A (2008) Analysis of pairwise cell interactions using an integrated dielectrophoretic-microfluidic system. Mol Syst Biol 4: 232.
15. Nash G F, Walsh D C, Kakkar A K (2001) The role of the coagulation system in tumour angiogenesis. Lancet Oncol 2(10): 608-613.
16. Crola Da Silva C, Lamerant-Fayel N, Paprocka M, Mitterrand M, Gosset D et al. (2009) Selective human endothelial cell activation by chemokines as a guide to cell homing. Immunology 126(3): 394-404.
17. Bielenberg D R, Pettaway C A, Takashima S, Klagsbrun M (2006) Neuropilins in neoplasms: expression, regulation, and function Exp Cell Res 312(5): 584-593.
18. Favier B, Alam A, Barron P, Bonnin J, Laboudie P et al. (2006) Neuropilin-2 interacts with VEGFR-2 and VEGFR-3 and promotes human endothelial cell survival and migration. Blood 108(4): 1243-1250.
19. Staton C A, Kumar I, Reed M W, Brown N J (2007) Neuropilins in physiological and pathological angiogenesis. J Pathol 212(3): 237-248.
20. Caunt M, Mak J, Liang W C, Stawicki S, Pan Q et al. (2008) Blocking neuropilin-2 function inhibits tumor cell metastasis. Cancer Cell 13(4): 331-342.
21. Klagsbrun M, Takashima S, Mamluk R (2002) The role of neuropilin in vascular and tumor biology. Adv Exp Med Biol 515: 33-48.
22. Bielenberg D R, Hida Y, Shimizu A, Kaipainen A, Kreuter M et al. (2004) Semaphorin 3F, a chemorepulsant for endothelial cells, induces a poorly vascularized, encapsulated, nonmetastatic tumor phenotype. J Clin Invest 114(9): 1260-1271.
23. Chen C, Li M, Chai H, Yang H, Fisher W E et al. (2005) Roles of neuropilins in neuronal development, angiogenesis, and cancers. World J Surg 29(3): 271-275.
24. Chabbert-de Ponnat I, Buffard V, Leroy K, Bagot M, Bensussan A et al. (2006) Antiproliferative effect of semaphorin 3F on human melanoma cell lines. J Invest Dermatol 126(10): 2343-2345.
25. Ellis L M (2006) The role of neuropilins in cancer. Mol Cancer Ther 5(5): 1099-1107.
26. Guttmann-Raviv N, Kessler 0, Shraga-Heled N, Lange T, Herzog Y et al. (2006) The neuropilins and their role in tumorigenesis and tumor progression. Cancer Lett 231(1): 1-11.
27. Bielenberg D R, Klagsbrun M (2007) Targeting endothelial and tumor cells with semaphorins. Cancer Metastasis Rev 26(3-4): 421-431.
28. Nasarre P, Constantin B, Rouhaud L, Harnois T, Raymond G et al. (2003) Semaphorin SEMA3F and VEGF have opposing effects on cell attachment and spreading. Neoplasia 5(1): 83-92.
29. Nasarre P, Kusy S, Constantin B, Castellani V, Drabkin H A et al. (2005) Semaphorin SEMA3F has a repulsing activity on breast cancer cells and inhibits E-cadherin-mediated cell adhesion. Neoplasia 7(2): 180-189.
30. Cohen T, Herzog Y Brodzky A, Greenson J K, Eldar S, Gluzman-Poltorak Z, Neufeld G Resnick M B. Neuropilin-2 is a novel marker expressed in pancreatic islet cells and endocrine pancreatic tumours. The Journal of pathology 2002; 198:77-82.

We claim:

1. A method for detecting or diagnosing melanoma in a subject comprising:
a. obtaining a biological sample from the subject;
b. detecting the presence of i) cells expressing neuropilin-2, ii) soluble neuropilin-2, or iii) a soluble fragment of neuropilin-2 in the sample; and
c. correlating the expression of neuropilin-2 with the presence of melanoma, thereby detecting or diagnosing melanoma in a subject.

2. The method of claim 1, wherein the method determines whether the subject is at risk for developing melanoma.

3. The method of claim 1, wherein the method predicts the recurrence of melanoma in the subject.

4. The method of claim 1, wherein the biological sample is tissue, a tissue homogenate, a tissue slice, a cell, a necropsy sample, a pathology sample, a biopsy sample, bodily fluid, blood, plasma, serum, urine, effusion, or spinal fluid.

5. The method of claim 1, wherein the detecting comprises performing an immunoassay, affinity column separation, magnetic selection, or FACS.

6. The method of claim 1, wherein the detecting comprises contacting the biological sample with an agent that selectively detects cells expressing neuropilin-2.

7. The method of claim 6, wherein the agent is an antibody that specifically binds to neuropilin-2.

8. The method of claim 7, wherein the antibody is labeled.

9. The method of claim 8, wherein the label is a fluorescent label, a moiety that binds another reporter ion, a magnetic particle, a heavy ion, a gold particle, or a quantum dot.

10. The method of claim 1, further comprising providing treatment for melanoma to a subject identified as expressing neuropilin-2.

11. A method of identifying melanoma cells in vivo in a subject comprising:
a. administering to the subject a diagnostically effective amount of an agent that selectively detects neuropilin-2, wherein the agent is labeled; and
b. detecting the labeled agent.

12. The method of claim 11, wherein the agent is an antibody that specifically binds to neuropilin-2.

13. A method of selectively isolating at least one target melanoma cell from a cell sample comprising:
a. providing the cell sample comprising at least one target melanoma cell; and
b. contacting the cell sample with an agent that selectively detects cells expressing neuropilin-2,
under conditions effective for the target melanoma cell(s) to bind to the agent, resulting in bound melanoma cell(s).

14. The method of claim 13, wherein the agent is an antibody that specifically binds to neuropilin-2.

15. The method of claim 13, further comprising using the isolated target melanoma cell(s) in an in vitro assay.

16. A method of treating a subject for melanoma comprising:
a. identifying a subject suffering from or at risk for developing melanoma; and
b. administering to the subject an agent that inhibits melanoma cell proliferation, wherein the agent inhibits neuropilin-2 mediated cell proliferation.

17. The method of claim 16, wherein the agent is a molecule that selectively binds to neuropilin-2.

18. The method of claim 16, wherein the agent is an antibody that specifically binds to neuropilin-2.

19. A kit for diagnosing or detecting melanoma in a subject, comprising at least one agent that detects the presence of i) cells expressing neuropilin-2, ii) soluble neuropilin-2, or iii) a soluble fragment of neuropilin-2, wherein the agent is an antibody that specifically binds to neuropilin-2 or a soluble fragment of neuropilin-2.

20. The kit of claim 19, wherein the kit is used to determine whether the subject is at risk for melanoma or to predict the recurrence of melanoma in the subject.
